SEQUENCE LISTING (44 sequences)

SEQ ID NO: 1 - protein, 727 aa, Artificial sequence
  MBP-PTE  wild type like S5 amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFITNSGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRGGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDSVNPDGMAFIPLRVIPFLREKGVPQETLAGITVTNPARFLSPTLRAS

SEQ ID NO: 2 - DNA, 2187 bp, Artificial sequence
  MBP-PTE  wild type like S5 nucleic acid sequence
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcatcaccaacagcggcgatcggatcaataccgtgcgcggtcctatcacaatctctgaagcgggtttcacactgactcacgagcacatctgcggcagctcggcaggattcttgcgtgcttggccggagttcttcggtagccgcaaagccctagcggaaaaggctgtgagaggattgcgccgcgccagagcggctggcgtgcgaacgattgtcgatgtgtcgactttcgatatcggtcgcgatgtcagtttattggccgaggtttcgcgggctgccgacgttcatatcgtggcggcgaccggcttgtggttcgacccgccactttcgatgcgattgaggagtgtagaggaactcacacagttcttcctgcgtgagattcaatatggcatcgaagacaccggaattagggcgggcattatcaaggtcgcgaccacaggcaaggcgaccccctttcaggagttagtgttaagggcggccgcccgggccagcttggccaccggtgttccggtaaccactcacacggcagcaagtcagcgcggtggtgagcagcaggccgccatttttgagtccgaaggcttgagcccctcacgggtttgtattggtcacagcgatgatactgacgatttgagctatctcaccgccctcgctgcgcgcggatacctcatcggtctagaccacatcccgcacagtgcgattggtctagaagataatgcgagtgcatcagccctcctgggcatccgttcgtggcaaacacgggctctcttgatcaaggcgctcatcgaccaaggctacatgaaacaaatcctcgtttcgaatgactggctgttcgggttttcgagctatgtcaccaacatcatggacgtgatggatagcgtgaaccccgacgggatggccttcattccactgagagtgatcccattcctacgagagaagggcgtcccacaggaaacgctggcaggcatcactgtgactaacccggcgcggttcttgtcaccgaccttgcgggcgtcatgataa

SEQ ID NO: 3 - protein, 336 aa, Artificial sequence
  PTE  wild type like S5 amino acid sequence
ITNSGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRGGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDSVNPDGMAFIPLRVIPFLREKGVPQETLAGITVTNPARFLSPTLRAS

SEQ ID NO: 4 - DNA, 1017 bp, Artificial sequence
  PTE  wild type like S5 nucleic acid sequence
atgatcaccaacagcggcgatcggatcaataccgtgcgcggtcctatcacaatctctgaagcgggtttcacactgactcacgagcacatctgcggcagctcggcaggattcttgcgtgcttggccggagttcttcggtagccgcaaagccctagcggaaaaggctgtgagaggattgcgccgcgccagagcggctggcgtgcgaacgattgtcgatgtgtcgactttcgatatcggtcgcgatgtcagtttattggccgaggtttcgcgggctgccgacgttcatatcgtggcggcgaccggcttgtggttcgacccgccactttcgatgcgattgaggagtgtagaggaactcacacagttcttcctgcgtgagattcaatatggcatcgaagacaccggaattagggcgggcattatcaaggtcgcgaccacaggcaaggcgaccccctttcaggagttagtgttaagggcggccgcccgggccagcttggccaccggtgttccggtaaccactcacacggcagcaagtcagcgcggtggtgagcagcaggccgccatttttgagtccgaaggcttgagcccctcacgggtttgtattggtcacagcgatgatactgacgatttgagctatctcaccgccctcgctgcgcgcggatacctcatcggtctagaccacatcccgcacagtgcgattggtctagaagataatgcgagtgcatcagccctcctgggcatccgttcgtggcaaacacgggctctcttgatcaaggcgctcatcgaccaaggctacatgaaacaaatcctcgtttcgaatgactggctgttcgggttttcgagctatgtcaccaacatcatggacgtgatggatagcgtgaaccccgacgggatggccttcattccactgagagtgatcccattcctacgagagaagggcgtcccacaggaaacgctggcaggcatcactgtgactaacccggcgcggttcttgtcaccgaccttgcgggcgtcatgataa

SEQ ID NO: 5 - protein, 726 aa, Artificial sequence
  MBP-IVA1-m2p0  amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFITNSGDRINTVRGPITISEAGFTLTHEHIMGSSAGFLRAWPEFFGSRAALVEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNSASALLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 6 - DNA, 2184 bp, Artificial sequence
  MBP-IVA1-m2p0  nucleic acid sequence
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcatcaccaacagcggcgaccgtatcaacaccgtccgtggtccgatcaccatctctgaggcgggcttcaccctgactcacgaacacattatgggttctagcgcaggttttctgcgcgcttggccggagtttttcggttctcgtgctgctctggtggaaaaagcggttcgtggcctgcgtcgtgcgcgtgcggctggtgtgcgtaccatcgttgacgtttctaccttcgacgccggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaactctgcgtccgctctgctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 7 - protein, 727 aa, Artificial sequence
  MBP-1-3-D5  amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFITNSGDRINTVRGPITISEAGFTLTHEHICGSSAGFLRAWPEFFGSRAALVEKAVRGLRRARAAGVRTIVDVSTFDIGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASALLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 8 - DNA, 2187 bp, Artificial sequence
  MBP-1-3-D5  nucleic acid sequence
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcatcaccaacagcggcgaccgtatcaacaccgtccgtggtccgatcaccatctctgaggcgggcttcaccctgactcacgaacacatttgcggttctagcgcaggttttctgcgcgcttggccggagtttttcggttctcgtgctgctctggtggaaaaagcggttcgtggcctgcgtcgtgcgcgtgcggctggtgtgcgtaccatcgttgacgtttctaccttcgacattggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaacgcatctgcgtccgctctgctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 9 - protein, 727 aa, Artificial sequence
  MBP-10-2-C3 amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFITNSGDRINTVRGPITISEAGFTLTHEHICGSSAGFLRAWPEFFGSRAALMEKAVRGLRRARAAGVRTIVDVSTFDIGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 10 - DNA, 2187 bp, Artificial sequence
  MBP-10-2-C3 nucleic acid sequence
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcatcaccaacagcggcgaccgtatcaacaccgtccgtggtccgatcaccatctctgaggcgggcttcaccctgactcacgaacacatttgcggttctagcgcaggttttctgcgcgcttggccggagtttttcggttctcgtgctgctctgatggaaaaagcggttcgtggcctgcgtcgtgcgcgtgcggctggtgtgcgtaccatcgttgacgtttctaccttcgacattggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaacgcatctgcgtcctcctggctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 11 - protein, 727 aa, Artificial sequence
  MBP-10-1-D11 amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFITNSGDRINTVRGPITISEAGFTLTHEHICGSSAGFLRAWPEFFGSRAALMEKAVRGLRRARAAGVRTIVDVSTFDIGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNAMASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 12 - DNA, 2187 bp, Artificial sequence
  MBP-10-1-D11 nucleic acid sequence
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcatcaccaacagcggcgaccgtatcaacaccgtccgtggtccgatcaccatctctgaggcgggcttcaccctgactcacgaacacatttgcggttctagcgcaggttttctgcgcgcttggccggagtttttcggttctcgtgctgctctgatggaaaaagcggttcgtggcctgcgtcgtgcgcgtgcggctggtgtgcgtaccatcgttgacgtttctaccttcgacattggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaacgcaatggcgtcctcttggctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 13 - protein, 727 aa, Artificial sequence
  MBP-10-1-D11-I106A amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFITNSGDRINTVRGPITISEAGFTLTHEHICGSSAGFLRAWPEFFGSRAALMEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNAMASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 14 - DNA, 2187 bp, Artificial sequence
  MBP-10-1-D11I106A nucleic acid sequence
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcatcaccaacagcggcgaccgtatcaacaccgtccgtggtccgatcaccatctctgaggcgggcttcaccctgactcacgaacacatttgcggttctagcgcaggttttctgcgcgcttggccggagtttttcggttctcgtgctgctctgatggaaaaagcggttcgtggcctgcgtcgtgcgcgtgcggctggtgtgcgtaccatcgttgacgtttctaccttcgacgccggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaacgcaatggcgtcctcctggctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 15 - protein, 335 aa, Artificial sequence
  IVA1-m2p0  amino acid sequence
ITNSGDRINTVRGPITISEAGFTLTHEHIMGSSAGFLRAWPEFFGSRAALVEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNSASALLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 16 - DNA, 1014 bp, Artificial sequence
  IVA1-m2p0  nucleic acid sequence
atgatcaccaacagcggcgaccgtatcaacaccgtccgtggtccgatcaccatctctgaggcgggcttcaccctgactcacgaacacattatgggttctagcgcaggttttctgcgcgcttggccggagtttttcggttctcgtgctgctctggtggaaaaagcggttcgtggcctgcgtcgtgcgcgtgcggctggtgtgcgtaccatcgttgacgtttctaccttcgacgccggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaactctgcgtccgctctgctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 17 - protein, 336 aa, Artificial sequence
  1-3-D5  amino acid sequence
ITNSGDRINTVRGPITISEAGFTLTHEHICGSSAGFLRAWPEFFGSRAALVEKAVRGLRRARAAGVRTIVDVSTFDIGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASALLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 18 - DNA, 1017 bp, Artificial sequence
  1-3-D5  nucleic acid sequence
atgatcaccaacagcggcgaccgtatcaacaccgtccgtggtccgatcaccatctctgaggcgggcttcaccctgactcacgaacacatttgcggttctagcgcaggttttctgcgcgcttggccggagtttttcggttctcgtgctgctctggtggaaaaagcggttcgtggcctgcgtcgtgcgcgtgcggctggtgtgcgtaccatcgttgacgtttctaccttcgacattggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaacgcatctgcgtccgctctgctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 19 - protein, 336 aa, Artificial sequence
  10-2-C3 amino acid sequence
ITNSGDRINTVRGPITISEAGFTLTHEHICGSSAGFLRAWPEFFGSRAALMEKAVRGLRRARAAGVRTIVDVSTFDIGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 20 - DNA, 1017 bp, Artificial sequence
  10-2-C3 nucleic acid sequence
atgatcaccaacagcggcgaccgtatcaacaccgtccgtggtccgatcaccatctctgaggcgggcttcaccctgactcacgaacacatttgcggttctagcgcaggttttctgcgcgcttggccggagtttttcggttctcgtgctgctctgatggaaaaagcggttcgtggcctgcgtcgtgcgcgtgcggctggtgtgcgtaccatcgttgacgtttctaccttcgacattggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaacgcatctgcgtcctcctggctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 21 - protein, 336 aa, Artificial sequence
  10-1-D11 amino acid sequence
ITNSGDRINTVRGPITISEAGFTLTHEHICGSSAGFLRAWPEFFGSRAALMEKAVRGLRRARAAGVRTIVDVSTFDIGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNAMASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 22 - DNA, 1017 bp, Artificial sequence
  10-1-D11 nucleic acid sequence
atgatcaccaacagcggcgaccgtatcaacaccgtccgtggtccgatcaccatctctgaggcgggcttcaccctgactcacgaacacatttgcggttctagcgcaggttttctgcgcgcttggccggagtttttcggttctcgtgctgctctgatggaaaaagcggttcgtggcctgcgtcgtgcgcgtgcggctggtgtgcgtaccatcgttgacgtttctaccttcgacattggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaacgcaatggcgtcctcttggctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 23 - protein, 336 aa, Artificial sequence
  10-1-D11-I106A amino acid sequence
ITNSGDRINTVRGPITISEAGFTLTHEHICGSSAGFLRAWPEFFGSRAALMEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNAMASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 24 - DNA, 1017 bp, Artificial sequence
  10-1-D11I106A nucleic acid sequence
atgatcaccaacagcggcgaccgtatcaacaccgtccgtggtccgatcaccatctctgaggcgggcttcaccctgactcacgaacacatttgcggttctagcgcaggttttctgcgcgcttggccggagtttttcggttctcgtgctgctctgatggaaaaagcggttcgtggcctgcgtcgtgcgcgtgcggctggtgtgcgtaccatcgttgacgtttctaccttcgacgccggtcgtgatgtttctctgctggccgaggtttctgaagcggccgatgttcacattgttgcagcgactggtctgtgggaagatccgccgctgtctatgcgtctgcgctctgttgaagaactcacccagttctttctccgtgaaatccagtacggcatcgaggacacgggtatccgtgccggtatcattaaagttgccaccaacggtaaagcgaccccgtttcaggaactggttctgcgtgcagcagctcgtgcctccctcgccaccggcgttccggtcaccacccacaccgacgcttctcagcgtgacggtgaacagcaggcggcgatcttcgaaagcgaaggtctggacccgtctcgtgtttgtatcggtcactctgacggcaccgatgatctggactacctgaccgcgctcgcggctcgtggttacctgattggcctggatggtattccgcactctgcgatcggcctcgaagacaacgcaatggcgtcctcctggctcggtaatcgctcttggcagacccgtgcgctgctgatcaaagcgctgatcgaccagggctacgttaaacagatcctggtttctaacgattggctgttcggtttttcttcttgggttaccaacatcatggacgttatggactctgttaacccagacggtatggcgttcatcccgctgcgtgttatcccgttcctgcgcgagaaaggtgttccacaagagacgctggcgaccatcaccgttgaaaaccctgctcgtttcctgtctccaaccctccgtgcttcttgataa

SEQ ID NO: 25 - protein, 727 aa, Artificial sequence
  MBP-1-3-D5-I106A amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFITNSGDRINTVRGPITISEAGFTLTHEHICGSSAGFLRAWPEFFGSRAALVEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASALLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 26 - protein, 336 aa, Artificial sequence
  1-3-D5-I106A amino acid sequence
ITNSGDRINTVRGPITISEAGFTLTHEHICGSSAGFLRAWPEFFGSRAALVEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASALLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 27 - protein, 727 aa, Artificial sequence
  MBP-10-2-C3-I106A + MBP amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFITNSGDRINTVRGPITISEAGFTLTHEHICGSSAGFLRAWPEFFGSRAALMEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 28 - protein, 336 aa, Artificial sequence
  10-2-C3-I106A amino acid sequence
ITNSGDRINTVRGPITISEAGFTLTHEHICGSSAGFLRAWPEFFGSRAALMEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 29 - protein, 366 aa, Artificial sequence
  MBPalone amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQ

SEQ ID NO: 30 - protein, 25 aa, Artificial sequence
  linker amino acid sequence
TNSSSNNNNNNNNNNLGIEGRISEF

SEQ ID NO: 31 - protein, 727 aa, Artificial sequence
  MBP-C23  amino acid sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFITNSGDRINTVRGPITISEAGFTLTHEHICGSSAGFLRAWPEFFGSRAALVEKAVRGLRRARAAGVRTIVDVSTFDIGRDVSLLAEVSRAADVHIVAATGLWEDPPLSMRLRSVEELTQFFLREIQYGIEDTGIRAGIIKVATNGKATPFQELVLRAAARASLATGVPVTTHTAASQRDGEQQAAIFESEGLSPSRVCIGHSDDTDDLSYLTALAARGYLIGLDGIPHSAIGLEDNASASALLGNRSWQTRALLIKALIDQGYMKQILVSNDWLFGFSSYVTNIMDVMDSVNPDGMAFIPLRVIPFLREKGVSQETLAGITVTNPARFLSPTLRAS

SEQ ID NO: 32 - DNA, 2187 bp, Artificial sequence
  MBP-C23  nucleic acid sequence
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcatcaccaacagcggcgatcggatcaataccgtgcgcggtcctatcacaatctctgaagcgggtttcacactgactcacgagcacatctgcggcagctcggcaggattcttgcgtgcttggccggagttcttcggtagccgcgcagccctagtggaaaaggctgtgagaggattgcgccgcgccagagcggctggcgtgcgaacgattgtcgatgtgtcgactttcgatatcggtcgcgatgtcagtttattggccgaggtttcgcgggctgccgacgttcatatcgtggcggcgaccggcttgtgggaggacccgccactttcgatgcggttgaggagtgtagaggaactcacacagttcttcctgcgtgagattcaatatggcatcgaagacaccggaattagggcgggcattatcaaggtcgcgaccaatggcaaggcgaccccctttcaggagttagtgttaagggcggccgcccgggccagcttggccaccggtgttccggtaaccactcacacggcagcaagtcagcgcgatggtgagcagcaggccgccatttttgagtccgaaggcttgagcccctcacgagtttgtattggtcacagcgatgatactgacgatttgagctatctcaccgccctcgctgcgcgcggatacctcatcggtctagacggcatcccgcacagtgcgattggtctagaagataatgcgagtgcatcagccctcctgggcaaccgttcgtggcaaacacgggctctcttgatcaaggcgctcatcgaccaaggctacatgaaacaaatcctcgtttcgaatgactggctgttcgggttttcgagctatgtcaccaacatcatggacgtgatggatagcgtgaaccccgacgggatggccttcattccactgagagtgatcccattcctacgagagaagggcgtctcacaggaaacgctggcaggcatcactgtgaccaacccggcgcggttcttgtcaccgaccttgcgggcgtcatgataa

SEQ ID NO: 33 - protein, 336 aa, Artificial sequence
  C23  amino acid sequence
ITNSGDRINTVRGPITISEAGFTLTHEHICGSSAGFLRAWPEFFGSRAALVEKAVRGLRRARAAGVRTIVDVSTFDIGRDVSLLAEVSRAADVHIVAATGLWEDPPLSMRLRSVEELTQFFLREIQYGIEDTGIRAGIIKVATNGKATPFQELVLRAAARASLATGVPVTTHTAASQRDGEQQAAIFESEGLSPSRVCIGHSDDTDDLSYLTALAARGYLIGLDGIPHSAIGLEDNASASALLGNRSWQTRALLIKALIDQGYMKQILVSNDWLFGFSSYVTNIMDVMDSVNPDGMAFIPLRVIPFLREKGVSQETLAGITVTNPARFLSPTLRAS

SEQ ID NO: 34 - DNA, 1017 bp, Artificial sequence
  C23  nucleic acid sequence
atgatcaccaacagcggcgatcggatcaataccgtgcgcggtcctatcacaatctctgaagcgggtttcacactgactcacgagcacatctgcggcagctcggcaggattcttgcgtgcttggccggagttcttcggtagccgcgcagccctagtggaaaaggctgtgagaggattgcgccgcgccagagcggctggcgtgcgaacgattgtcgatgtgtcgactttcgatatcggtcgcgatgtcagtttattggccgaggtttcgcgggctgccgacgttcatatcgtggcggcgaccggcttgtgggaggacccgccactttcgatgcggttgaggagtgtagaggaactcacacagttcttcctgcgtgagattcaatatggcatcgaagacaccggaattagggcgggcattatcaaggtcgcgaccaatggcaaggcgaccccctttcaggagttagtgttaagggcggccgcccgggccagcttggccaccggtgttccggtaaccactcacacggcagcaagtcagcgcgatggtgagcagcaggccgccatttttgagtccgaaggcttgagcccctcacgagtttgtattggtcacagcgatgatactgacgatttgagctatctcaccgccctcgctgcgcgcggatacctcatcggtctagacggcatcccgcacagtgcgattggtctagaagataatgcgagtgcatcagccctcctgggcaaccgttcgtggcaaacacgggctctcttgatcaaggcgctcatcgaccaaggctacatgaaacaaatcctcgtttcgaatgactggctgttcgggttttcgagctatgtcaccaacatcatggacgtgatggatagcgtgaaccccgacgggatggccttcattccactgagagtgatcccattcctacgagagaagggcgtctcacaggaaacgctggcaggcatcactgtgaccaacccggcgcggttcttgtcaccgaccttgcgggcgtcatgataa

SEQ ID NO: 35 - protein, 29 aa, Artificial sequence
  first 29 amino acids sequence of wild-type PTE
MQTRRVVLKSAAAAGTLLGGLAGCASVAG

SEQ ID NO: 36 - protein, 365 aa, Artificial sequence
  PTE Full length for numbering
MQTRRVVLKSAAAAGTLLGGLAGCASVAGSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 37 - protein, 332 aa, Artificial sequence
  PTE  wild type like S5 amino acid sequence
GDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRGGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDSVNPDGMAFIPLRVIPFLREKGVPQETLAGITVTNPARFLSPTLRAS

SEQ ID NO: 38 - protein, 331 aa, Artificial sequence
  IVA1-m2p0  amino acid sequence
GDRINTVRGPITISEAGFTLTHEHIMGSSAGFLRAWPEFFGSRAALVEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNSASALLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 39 - protein, 332 aa, Artificial sequence
  1-3-D5  amino acid sequence
GDRINTVRGPITISEAGFTLTHEHICGSSAGFLRAWPEFFGSRAALVEKAVRGLRRARAAGVRTIVDVSTFDIGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASALLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 40 - protein, 332 aa, Artificial sequence
  10-2-C3 amino acid sequence
GDRINTVRGPITISEAGFTLTHEHICGSSAGFLRAWPEFFGSRAALMEKAVRGLRRARAAGVRTIVDVSTFDIGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 41 - protein, 332 aa, Artificial sequence
  10-1-D11 amino acid sequence
GDRINTVRGPITISEAGFTLTHEHICGSSAGFLRAWPEFFGSRAALMEKAVRGLRRARAAGVRTIVDVSTFDIGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNAMASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 42 - protein, 332 aa, Artificial sequence
  10-1-D11-I106A amino acid sequence
GDRINTVRGPITISEAGFTLTHEHICGSSAGFLRAWPEFFGSRAALMEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNAMASSWLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 43 - protein, 332 aa, Artificial sequence
  1-3-D5-I106A amino acid sequence
GDRINTVRGPITISEAGFTLTHEHICGSSAGFLRAWPEFFGSRAALVEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASALLGNRSWQTRALLIKALIDQGYVKQILVSNDWLFGFSSWVTNIMDVMDSVNPDGMAFIPLRVIPFLREKGVPQETLATITVENPARFLSPTLRAS

SEQ ID NO: 44 - protein, 332 aa, Artificial sequence
  10-2-C3-I106A amino acid sequence
GDRINTVRGPITISEAGFTLTHEHICGSSAGFLRAWPEFFGSRAALMEKAVRGLRRARAAGVRTIVDVSTFDAGRDVSLLAEVSEAADVHIVAATGLWEDPPLSMRLRSVEELTQFFLREIQYGIEDTGIRAGIIKVATNGKATPFQELVLRAAARASLATGVPVTTHTDASQRDGEQQAAIFESEGLDPSRVCIGHSDGTDDLDYLTALAARGYLIGLDGIPHSAIGLEDNASASSWLGNRSWQTRALLIKALIDQGYVKQILVSNDWLFGFSSWVTNIMDVMDSVNPDGMAFIPLRVIPFLREKGVPQETLATITVENPARFLSPTLRAS

(19) United States
(12) Patent Application Publication
(10) Pub. No.: US 2019/0359956 A1
(43) Pub. Date: Nov. 28, 2019

(54) PHOSPHOTRIESTERASES FOR TREATING OR PREVENTING ORGANOPHOSPHATE EXPOSURE ASSOCIATED DAMAGE

(71) Applicant: Yeda Research and Development Co. Ltd., Rehovot (IL)

(72) Inventors: Dan S. TAWFIK, Jerusalem (IL); Moshe GOLDSMITH, Rehovot (IL); Yaacov ASHANI, Rehovot (IL); Nidhi AGGARWAL, Rehovot (IL)

(73) Assignee: Yeda Research and Development Co. Ltd., Rehovot (IL)

(21) Appl. No.: 16/348,516

(22) PCT Filed: Nov. 9, 2017
(86) PCT No.: PCT/IL2017/051220

§ 371 Date: May 9, 2019

Related U.S. Application Data
(60) Provisional application No. 62/419,980, filed on Nov. 10, 2016.

(51) Int. Cl. C12N 9/16; A61K 38/46; A61K 9/00

(52) U.S. Cl. CPC C12N 9/16; A61K 38/465; C12Y 301/01007; C12Y 301/08001; A61K 9/0014

(57) ABSTRACT

Polypeptides are disclosed which comprise an amino acid sequence of phosphotriesterase (PTE) having enhanced catalytic efficiency for VX-type or RVX-type nerve agents. Uses thereof are also disclosed.

### FIELD AND BACKGROUND OF THE INVENTION

The present invention, in some embodiments thereof, relates to phosphotriesterase (PTE) enzymes capable of hydrolyzing nerve agents.

At present, both prophylaxis and post-intoxication treatments of chemical warfare nerve agent (CWNA) poisoning are based on drugs selected to counteract the symptoms caused by accumulation of acetylcholine in cholinergic neurons. Current antidotal régimes consist of pretreatment with pyridostigmine, and of post-exposure therapy that involves administration of a cocktail containing atropine, an oxime reactivator and an anticonvulsant drug such as diazepam. The multi-drug approach against CWNA toxicity has been adopted by many countries and integrated into their civil and military medical protocols. However, it is commonly recognized that these drug régimes suffer from several disadvantages that call for new therapeutic strategies. The preferred approach is to rapidly detoxify the CWNA in the blood before it has had the chance to reach its physiological targets. One way of achieving this objective is by the use of bioscavengers. However, use of the best stoichiometric bioscavenger currently available (human butyrylcholinesterase, hBChE) requires administration of hundreds of milligrams of protein to confer protection against toxic doses of CWNA. A safer and more effective treatment strategy can be achieved by using a catalytic bioscavenger to rapidly degrade the intoxicating OP in the circulation.

The promiscuous nerve-agent hydrolyzing activities of the enzyme phosphotriesterase (PTE) make it a prime candidate both for prophylactic and post exposure treatment of nerve-agent intoxications. However, efficient in-vivo detoxification using low doses of enzymes (≤50 mg/70 kg) following exposure to toxic doses of nerve agents, requires that the catalytic efficiencies (k_{cat}/K_{M}) of wild-type PTE towards the toxic nerve agent isomers will be increased.

PTE variants that can efficiently hydrolyze V-type nerve agents are disclosed in Cherney et al, 2013, ACS Chem Biol 8: 2394-2403. In-vivo post-exposure activity of one of these variants (C23) was demonstrated in guinea-pigs intoxicated with a lethal dose of VX (Worek et al, 2014, Toxicol Lett 231: 45-54).

Additional background art includes U.S. Pat. No. 8,735,124 and WO2016/092555.

### SUMMARY OF THE INVENTION

According to an aspect of the present invention, there is provided a polypeptide comprising an amino acid sequence of phosphotriesterase (PTE), wherein the amino acid sequence comprises the mutations K77A, A80V/M, F132E, T173N, G208D, D233G/N/M/L/R/V/A/S/T, H254G, I274N and Y309W, wherein the numbering of the mutations is according to PDB 1HZY crystal structure numbering, wherein the polypeptide has at least 2500 fold the catalytic efficiency for a VX-type nerve agent as a polypeptide which consists of the sequence as set forth in SEQ ID NO: 1, when assayed at 25° C. under identical conditions and at least 3000 fold the catalytic efficiency for a RVX-type nerve agent as a polypeptide which consists of the sequence as set forth in SEQ ID NO: 1, when assayed at 25° C. under identical conditions.

According to an aspect of the present invention, there is provided a polypeptide comprising an amino acid sequence of phosphotriesterase (PTE) having at least 8,000 fold the catalytic efficiency for a RVX-type nerve agent as a polypeptide which consists of the sequence as set forth in SEQ ID NO: 1, when assayed at 25° C. under identical conditions, wherein the amino acid sequence comprises the mutations K77A, A80V/M, I106A, F132E, T173N, G208D, H254G, I274N, Y309W and D233G/N/M/L/R/V/A/S/T, wherein the numbering of the mutations is according to PDB 1HZY crystal structure numbering.

According to an aspect of the present invention, there is provided a polypeptide comprising an amino acid sequence at least 99% homologous to the sequence as set forth in SEQ ID NO: 5, 7, 9, 11, 13, 15, 17, 19, 21, 23, 25, 26, 27, 28, 38, 39, 40, 41, 42, 43 or 44.

According to an aspect of the present invention, there is provided a polypeptide comprising the amino acid sequence as set forth in SEQ ID NO: 5, 7, 9, 11, 13, 15, 17, 19, 21, 23, 25, 26, 27, 28, 38, 39, 40, 41, 42, 43 or 44.

According to an aspect of the present invention, there is provided an isolated polynucleotide comprising a nucleic acid sequence encoding any of the polypeptides disclosed herein.

According to an aspect of the present invention, there is provided a pharmaceutical composition comprising as an active ingredient any of the polypeptides disclosed herein, and a pharmaceutically acceptable carrier.

According to an aspect of the present invention, there is provided a nucleic acid construct comprising the isolated polynucleotide disclosed herein and a cis-regulatory element driving expression of the polynucleotide.

According to an aspect of the present invention, there is provided a method of treating an organophosphate exposure associated damage in a subject, comprising administering to the subject a therapeutically effective amount of any of the polypeptides disclosed herein.

According to an aspect of the present invention, there is provided an article of manufacture for treating or preventing organophosphate exposure associated damage, the article of manufacture comprising any of the polypeptides disclosed herein immobilized on to a solid support.

According to an aspect of the present invention, there is provided a method of detoxifying a surface, the method comprising contacting the surface with any of the polypeptides disclosed herein, thereby detoxifying the surface.

According to embodiments of the present invention, the amino acid sequence comprises the mutation D233G.

According to embodiments of the present invention, the amino acid at position 106 is isoleucine.

According to embodiments of the present invention, the polypeptide has at least 10 fold the catalytic efficiency for a VX-type nerve agent as a polypeptide which consists of the sequence as set forth in SEQ ID NO: 31, when assayed at 25° C. under identical conditions.

According to embodiments of the present invention, the polypeptide comprises the mutations A80M, A270S, L271W and D233G.

According to embodiments of the present invention, the polypeptide comprises a PTE amino acid sequence at least 99% homologous to the sequence as set forth in SEQ ID NO: 9, 19 or 40.

According to embodiments of the present invention, the polypeptide comprises the mutations A80M, S267M, A270S, D233G and L271W.

According to embodiments of the present invention, the polypeptide comprises a PTE amino acid sequence at least 99% homologous to the sequence as set forth in SEQ ID NOs: 11, 21 or 41.

According to embodiments of the present invention, the polypeptide comprises the mutations K77A, A80V, F132E, T173N, G208D, D233G, H254G, I274N and Y309W.

According to embodiments of the present invention, the polypeptide comprises a PTE amino acid sequence at least 99% homologous to the sequence as set forth in SEQ ID NOs: 7, 17 or 39.

According to embodiments of the present invention, the polypeptide further comprises the mutations A80V, C59M/V/F and an A266 deletion.

According to embodiments of the present invention, the polypeptide further comprises the mutations A80V, C59M and an A266 deletion.

According to embodiments of the present invention, the polypeptide comprises a PTE amino acid sequence at least 99% homologous to the sequence as set forth in SEQ ID NOs: 5, 15 or 38.

According to embodiments of the present invention, the polypeptide further comprises the mutations A80M, S267M, A270S and L271W.

According to embodiments of the present invention, the polypeptide comprises a PTE amino acid sequence at least 99% homologous to the sequence as set forth in 13, 23 or 42.

According to embodiments of the present invention, the polypeptide further comprises at least one of the stabilizing mutations selected from the group consisting of R118E, A203D, S222D, S238D, M293V/A, G348T and T352E/D.

According to embodiments of the present invention, the polypeptide further comprises each of the stabilizing mutations R118E, A203D, S222D, S238D, M293V, G348T and T352E/D.

According to embodiments of the present invention, the polypeptide is expressible in bacteria.

According to embodiments of the present invention, the polypeptide comprises an amino acid sequence at least 99% homologous to the sequence as set forth in SEQ ID NO: 7, 17, 9, 19, 11, 21, 39, 40 or 41.

According to embodiments of the present invention, the polypeptide comprises an amino acid sequence at least 99% homologous to the sequence as set forth in SEQ ID NO: 5, 15, 13, 23, 25, 26, 27, 28, 38, 42, 43 or 44.

According to embodiments of the present invention, the polypeptide consists of the amino acid sequence as set forth in SEQ ID NOs: 5, 7, 9, 11, 13, 25 and 27.

According to embodiments of the present invention, the solid support is for topical administration.

According to embodiments of the present invention, the solid support for topical administration is selected from the group consisting of a sponge, a wipe and a fabric.

According to embodiments of the present invention, the solid support is selected from the group consisting of a filter, a fabric and a lining.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a scheme of the screening assay. V-type nerve agents react either with acetylcholinesterase (AChE) to give the covalently inhibited enzyme (left arrow) or may be hydrolyzed by PTE (right arrow). The latter prevents the inhibition of AChE. The uninhibited AChE hydrolyzes the subsequently added substrate acetylthiocholine (5), thereby releasing thio-acetylcholine (7) that can then be detected by a reaction with DTNB; 5,5-Dithiobis(2-nitrobenzoic acid).

FIG. 2 illustrates the optimization of PTE's catalytic efficiency (k_{cat}/K_{M}) for hydrolysis of Sₚ-VX. The catalytic efficiency of the most active variant from each round of directed evolution is indicated. Round #0 denotes the starting point, a wild-type-like variant dubbed PTE S5. The dashed red line marks PTE-C23, the end of our previous directed evolution effort, and the subsequent rounds (6-13) are described here. The black arrow marks the introduction of the computationally stabilized variant C23-Y309W-m2p0. Error bars denote the standard deviation of the catalytic efficiency values.

FIGS. 3A-B illustrate the thermal stability and metal binding of PTE variants. FIG. 3A. Thermal inactivation assay. Lysates of cells expressing wt-like PTE S5, variant C23-Y309W, and stabilized variant C23-Y309W-m2p0 were incubated at different temperatures (45-70° C.) for 30 min in replicates (12 per variant). The residual paraoxoanase activity was measured after cooling down to room temperature. Shown is the % residual activity (normalized to the activity of untreated enzyme) as a function of the incubation temperature. The inactivation mid-temperature, T_{1/2}, of purified PTE-S5 (Goldenzweig, et al., 2016) was similar to that measured in crude lysate. FIG. 3B. Metal chelator inactivation assay. Crude lysates of E. coli cells in which the above variants were expressed were incubated for 30 min at 37° C. with a zinc chelator (50 μM 1,10-phenanthroline). The residual paraoxonase activities are plotted relative to the activity in a zinc containing buffer.

FIG. 4: Catalytic rate tradeoffs between VX and RVX. Plotted are the catalytic efficiencies (k_{cat}/K_{M}) of representative variants from different rounds of directed evolution with the Sₚ isomers of VX (blue bars) and RVX (red, stripped bars). The round number is presented at the bottom of the horizontal axis. Round 0 denotes the wt-like PTE-S5. Evolved variants derived from library screens are show left to the vertical dashed line, and engineered I106A mutants are shown on its right. Data for PTE-S5 and C23 are from (Cherny, et al., 2013).

FIG. 5 illustrates temperature inactivation assay for C23-Y309W stabilized designs. The residual paraoxonase activity of three purified, PROSS designed proteins (C23_m0p45, C23_m2p0 and C23_m0p95) was measured following a 30 minute incubation period at 50° C. (first column of each mutant) and 60° C. (the second column for each mutant). The remaining activity was measured relative to room temp' activity.

FIG. 6 is a bar graph illustrating a metal chelator inactivation assay for C23-Y309W stabilized designs. The residual paraoxonase activity of three purified, PROSS designed proteins (C23_m0p45, C23_m2p0 and C23_m0p95) was measured following a 30 min incubation period with 50 μM 1,10 phenanthroline. The remaining activity was measured relative to the activity of the variants without phenanthrolin.

FIG. 7 illustrates catalytic efficiency tradeoffs with mutations at position 106. The ratio of hydrolytic efficiency of VX to RVX is depicted for the best variants from round 13 and for the best RVX variant from round 12 (IVA1-m2p0). Horizontal axis—name of variant and substitution of position 106 (if not indicated, it is an Ile).

FIG. 8 illustrates hydrolysis of Sₚ-VX and Sₚ—RVX by variant 10-1-D11-I106A. A time-course of the hydrolysis of Sₚ-VX (20 μM, blue circles) and Sₚ—RVX (10 μM; red squares) in the presence of PTE variant 10-1-D11-I106A (0.036 μM with Sₚ-VX; 0.018 μM with Sₚ-RVX). Substrate hydrolysis was monitored at 412 nm using DTNB (see materials and methods). Data points were fitted to a mono-exponential association curve.

### DESCRIPTION OF SPECIFIC EMBODIMENTS OF THE INVENTION

The present invention, in some embodiments thereof, relates to phosphotriesterase (PTE) enzymes capable of hydrolyzing nerve agents and, more particularly, but not exclusively, to V-type nerve agents.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The promiscuous nerve-agent hydrolyzing activities of the enzyme phosphotriesterase (PTE) make it a prime candidate both for prophylactic and post exposure treatment of nerve-agent intoxications. However, efficient in-vivo detoxification using low doses of enzymes (≤50 mg/70 kg) following exposure to toxic doses of nerve agents requires that their catalytic efficiencies (k_{cat}/K_{M}) towards the toxic nerve agent isomers will be greater than 1×10⁷ M⁻¹ min⁻¹.

Previously, the present inventors generated PTE variants whose activity against the three major V-type nerve agents (VX, RVX and CVX) was improved relative to wt PTE (Cherny et al, 2013, ACS Chem Biol 8: 2394-2403 and WO2016/092555).

The present inventors sought to identify additional enhancement of the catalytic activities of PTE in order to obtain variants that could serve as lead compounds for further drug development and have now found that additional mutations at positions 233 carried out on the previously disclosed C23 mutant (Cherny et al, 2013, ACS Chem Biol 8: 2394-2403) increased its catalytic activity towards VX type nerve agent by up to 10 fold. Furthermore, the mutants obtained had broad spectrum activity having at least 3000 fold activity for the RVX relative to wt PTE.

Using the M2p0 mutant (as disclosed in WO2016/092555) as a starting point, additional mutants were generated, including those having a mutation at position 59, which had enhanced catalytic activities towards RVX and/or enhanced stabilities.

Thus, according to one aspect of the invention there is provided a polypeptide comprising an amino acid sequence of phosphotriesterase (PTE), wherein the amino acid sequence comprises the mutations K77A, A80V/M, F132E, T173N, G208D, D233G/N/M/L/R/V/A/S/T, H254G, I274N and Y309W, wherein the numbering of the mutations is according to PDB 1HZY crystal structure numbering, wherein the polypeptide has at least 2500 fold the catalytic efficiency for a VX-type nerve agent as a polypeptide which consists of the sequence as set forth in SEQ ID NO: 1, when assayed at 25° C. under identical conditions and at least 3000 fold the catalytic efficiency for a RVX-type nerve agent as a polypeptide which consists of the sequence as set forth in SEQ ID NO: 1, when assayed at 25° C. under identical conditions.

As used herein, the term “phosphotriesterase” abbreviated herein to PTE, also referred to as Parathion hydrolase (EC: 3.1.8.1), refers to an enzyme belonging to the amidohydrolase superfamily. The phosphotriesterases of this aspect of the present invention are bacterial phosphotriesterases that have an enhanced catalytic activity towards V-type organophosphonates due to an extended loop 7 amino acid sequence, as compared to other phosphotriesterases. Such phosphotriesterases have been identified in Brevundimonas diminuta, Flavobacterium sp. (PTEflavob) and Agrobacterium sp.

According to a particular embodiment, the phosphotriesterase of this aspect of the present invention comprises at least 100 consecutive amino acids of the native sequence of the B. diminuta PTE, at least 150 consecutive amino acids of the native sequence of the B. diminuta PTE, at least 200 consecutive amino acids of the native sequence of the B. diminuta PTE, at least 250 consecutive amino acids of the native sequence of the B. diminuta PTE, at least 300 consecutive amino acids of the native sequence of the B. diminuta PTE, at least 310 consecutive amino acids of the native sequence of the B. diminuta PTE, at least 320 consecutive amino acids of the native sequence of the B. diminuta PTE, at least 321 consecutive amino acids of the native sequence of the B. diminuta PTE, at least 322 consecutive amino acids of the native sequence of the B. diminuta PTE, at least 323 consecutive amino acids of the native sequence of the B. diminuta PTE, at least 324 consecutive amino acids of the native sequence of the B. diminuta PTE. According to a particular embodiment, the phosphotriesterase does not include the first four amino acids of the native B. diminuta PTE (i.e. is devoid of the sequence SIGT). For the purpose of this invention, the term “consecutive amino acids” also includes the mutations which are disclosed herein.

As used herein, a “nerve agent” refers to an organophosphate (OP) compound such as having an acetylcholinesterase inhibitory activity. The toxicity of an OP compound depends on the rate of its inhibition of acetylcholinesterase with the concomitant release of the leaving group such as fluoride, alkylthiolate, cyanide or aryoxy group. The nerve agent may be a racemic composition or a purified enantiomer (e.g., Sp or Rp). According to this aspect of the present invention the nerve agent is a V-type nerve agent (e.g., VX, CVX or RVX).

Methods of measuring catalytic efficiency of the variants described herein are known in the art and include for example measuring the release of thiol leaving group using the Ellman's reagent dithionitrobenzoic acid (DTNB) following incubation with the PTE variants, or by measuring the rate of loss of AChE inhibition upon incubation of racemic OPs with the PTE variants. Both of these methods are further described in the Examples section herein below.

Catalytic activities may be accurately compared when the same assay method is used, under the same experimental conditions (temperature, time etc.). In order to minimize differences in experimental conditions, it is preferable that the catalytic activities of the test polypeptide and control (e.g. SEQ ID NO: 1, also referred to herein as PTE-S5, or wild-type PTE, or SEQ ID NO: 31, also referred to herein as C23) are measured at substantially the same time (e.g. during the same experiment) and using the same stock solutions.

According to an embodiment of this aspect of the present invention, the test polypeptide has at least 2000, 2500, 3000, 3500, 4000, 4500 or 5000 times the catalytic efficiency for a VX-type nerve agent as a polypeptide which consists of the sequence as set forth in SEQ ID NO: 1, when assayed at 25° C., under identical conditions.

Furthermore, the test polypeptide has at least 2500, 3000, 3500, 4000, 4500 or 5000 times the catalytic efficiency for a RVX-type nerve agent as a polypeptide which consists of the sequence as set forth in SEQ ID NO: 1, when assayed at 25° C., under identical conditions.

Methods of selecting PTE polypeptides with the desired activity are provided in the Examples section below. Typically, these methods involve directed evolution of PTE using structure-based as well as random mutagenesis, and combining low-throughput methodologies (96-well plate screening) with high-throughput screens e.g., using compartmentalization in emulsions.

As used herein the phrase “in vitro evolution process” (also referred to as “a directed evolution process”) refers to the mutagenesis and/or recombination of genes and selection or screening of a desired activity. A number of methods which can be utilized to effect in vitro evolution, are known in the art. One approach of executing the in-vitro evolution process is provided in the Examples section.

General outline of directed evolution is provided in Tracewell C A, Arnold F H “Directed enzyme evolution: climbing fitness peaks one amino acid at a time” Curr Opin Chem Biol. 2009 February; 13(1):3-9. Epub 2009 Feb. 25; Gerlt J A, Babbitt P C, Curr Opin Chem Biol. 2009 February; 13(1):10-8. Epub 2009 Feb. 23 and WO2004/078991 (either of which is hereby incorporated by reference in its entirety).

Methods of producing recombinant proteins are well known in the art and are further described herein below.

The phosphotriesterases of this aspect of the present invention comprise mutations over wild-type phosphotriesterases which improve the hydrolytic efficiency of PTE to V-type nerve agent substrates. One of the mutations is at position 233.

The polypeptides of the present invention may be expressed in any expression system—e.g. yeast, insects, human cells, CHO cells, plant cells and bacteria cells. Additional expression systems are further described herein below. In a particular embodiment, the PTE polypeptides are expressed in bacteria such as E. coli [e.g., BL21, BL21 (DE3), Origami B (DE3), available from Novagen (www(dot)calbiochem(dot)com) and RIL (DE3) available from Stratagene, (www(dot)stratagene(dot)com). Essentially, at least 2%, at least 5%, at least 10%, at least 20%, at least 30%, at least 40%, at least 50%, at least 60%, at least 70%, at least 80%, at least 90%, at least 95% or more, say 100%, of bacterially expressed protein remains soluble (i.e., does not precipitate into inclusion bodies).

The present inventors have found that removal of the first 29 amino acids of the wild-type PTE aided in the successful expression in bacteria. It will be appreciated that for expression in other systems, the present inventors contemplate polypeptides comprising the sequences described herein together with the first 29 amino acids of wild-type PTE (SEQ ID NO: 35). In addition, at least the next 4, 5, 6, 7, 8, 9, 10, 11, 12, 13, 14, 15, 16, 17, 18, 19, 20 amino acids may be replaced as well. For example, for the presently disclosed polypeptides the sequence SIGT was replaced with the sequence ITNS.

The numbering of the mutations follows the PTE crystal structure numbering (PDB 1HZY) as set forth in SEQ ID NO: 36. Thus the first amino acid, i.e. methionine, of the sequence SEQ ID NO: 36 is considered position 1, the second amino acid, i.e. glutamine, of the sequence is position 2, the third amino acid, i.e. threonine, of the sequence is position 3 etc.

For polypeptides which are expressed in bacterial systems (where the first 29 amino acids have been removed), the first PTE amino acid in the sequences provided (Ile) (see sequences 15, 17, 19, 21, 23, 26, 28 and 37-44) is counted to be at the corresponding position 30 of SEQ ID NO: 36, the second amino acid (threonine) is counted to be at the corresponding position 31 of SEQ ID NO: 36, the third amino acid (asparagine) is counted to be at the corresponding position 32 of SEQ ID NO: 36 etc.

The present inventors have found that in order to enhance hydrolytic activity of the PTE polypeptides, the amino acid which naturally exists at position 233 (aspartic acid, D) may be mutated to any of glycine, asparagine, methionine, leucine, arginine, valine, alanine, serine or threonine.

According to a particular embodiment, the mutation is from aspartic acid to glycine (D233G).

Other essential mutations include K77A, A80V/M, F132E, T173N, G208D, H254G, I274N and Y309W.

It should be appreciated that amino acid coordinates can be adapted easily to PTE variants of the same or other species by amino acid sequence alignments which may be done manually or using specific bioinformatic tools such as FASTA, L-ALIGN and protein Blast.

According to a specific embodiment, mutations which may be employed to improve the hydrolytic efficiency of PTE to V-type nerve agent substrates comprise each of the mutations K77A, A80V, F132E, T173N, G208D, D233G, H254G, I274N and Y309W.

Polypeptides with the above described mutations may further comprise each of the following stabilizing mutations:

R118E, A203D, S222D, S238D, M293V/A, G348T and T352E/D.

PTE polypeptides which each of these mutations may have a catalytic efficiency of about 28×10⁶M⁻¹ for Sp-VX and about 2.5×10⁶M⁻¹ for Sp-RVX.

Thus, according to a specific embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 17 or 39 as determined using the Standard protein-protein BLAST [blastp] software of the NCBI, with the restriction that the amino acid at position 77 is A and not replaceable, the amino acid at position 80 is V or M (preferably V) and not replaceable, the amino acid at position 118 is E and not replaceable, the amino acid at position 132 is E and not replaceable, that the amino acid at position 173 is N and not replaceable, the amino acid at position 203 is D and not replaceable, the amino acid at position 208 is D and not replaceable, the amino acid at position 222 is D and not replaceable, the amino acid at position 238 is D and not replaceable, the amino acid at position 254 is G and not replaceable, the amino acid at position 274 is N and not replaceable, the amino acid at position 293 is V/A (most preferably V) and not replaceable, the amino acid at position 309 is W and not replaceable, the amino acid at position 348 is T and not replaceable and the amino acid at position 352 is E or D (most preferably E) and not replaceable. Furthermore, the amino acid at position 233 is any of G/N/M/L/R/V/A/S/T (most preferably G) and not any other amino acid. In one embodiment, the amino acid at position 106 is isoleucine and not replaceable. This polypeptide is referred to herein as 1-3-D5^{d}.

The present inventors have demonstrated that additional mutations beyond those described herein above (i.e. K77A, A80V/M, F132E, T173N, G208D, D233G, H254G, I274N and Y309W) may further enhance the hydrolytic efficiency of PTE to VX-type nerve agent substrates such that the hydrolytic activity for a VX-type nerve agent is about 10 fold the activity of C23 (SEQ ID NO: 31) for a VX-type nerve agent. This corresponds to an activity.

One exemplary polypeptide is the one having each of the mutations K77A, A80M, F132E, T173N, G208D, D233G, H254G, A270S, L271W, I274N and Y309W.

This polypeptide may further comprise each of the following stabilizing mutations: R118E, A203D, S222D, S238D, M293V/A, G348T and T352E/D.

PTE polypeptides which each of these mutations may have a catalytic efficiency of about 50×10⁶M⁻¹Min⁻¹ for Sp-VX and about 3.2×10⁶M⁻¹min⁻¹ for Sp-RVX.

Thus, according to a specific embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologus/identical to the sequence as set forth in SEQ ID NO: 19 or 40 as determined using the Standard protein-protein BLAST [blastp] software of the NCBI, with the restriction that the amino acid at position 77 is A and not replaceable, the amino acid at position 80 is V or M (preferably M) and not replaceable, the amino acid at position 118 is E and not replaceable, the amino acid at position 132 is E and not replaceable, that the amino acid at position 173 is N and not replaceable, the amino acid at position 203 is D and not replaceable, the amino acid at position 208 is D and not replaceable, the amino acid at position 222 is D and not replaceable, the amino acid at position 238 is D and not replaceable, the amino acid at position 254 is G and not replaceable, the amino acid at position 270 is S and not replaceable, the amino acid at position 271 is W and not replaceable, the amino acid at position 274 is N and not replaceable, the amino acid at position 293 is V/A (most preferably V) and not replaceable, the amino acid at position 309 is W and not replaceable, the amino acid at position 348 is T and not replaceable and the amino acid at position 352 is E or D (most preferably E) and not replaceable. Furthermore, the amino acid at position 233 is any of G/N/M/L/R/V/A/S/T (most preferably G) and not any other amino acid. In one embodiment, the amino acid at position 106 is isoleucine and not replaceable. This polypeptide is referred to herein as 10-2-C3d.

Another exemplary polypeptide is the one having each of the mutations K77A, A80M, F132E, T173N, G208D, D233G, H254G, S267M, A270S, L271W, I274N and Y309W.

This polypeptide may further comprise each of the following stabilizing mutations: R118E, A203D, S222D, S238D, M293V/A, G348T and T352E/D.

PTE polypeptides which each of these mutations may have a catalytic efficiency of about 51×10⁶M⁻¹min⁻¹ for Sp-VX and about 2.7×10⁶M⁻¹ min⁻¹ for Sp-RVX.

Thus, according to a specific embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 21 or 41 as determined using the Standard protein-protein BLAST [blastp] software of the NCBI, with the restriction that the amino acid at position 77 is A and not replaceable, the amino acid at position 80 is V or M (preferably M) and not replaceable, the amino acid at position 118 is E and not replaceable, the amino acid at position 132 is E and not replaceable, that the amino acid at position 173 is N and not replaceable, the amino acid at position 203 is D and not replaceable, the amino acid at position 208 is D and not replaceable, the amino acid at position 222 is D and not replaceable, the amino acid at position 238 is D and not replaceable, the amino acid at position 254 is G and not replaceable, the amino acid at position 267 is M and not replaceable, the amino acid at position 270 is S and not replaceable, the amino acid at position 271 is W and not replaceable, the amino acid at position 274 is N and not replaceable, the amino acid at position 293 is V/A (most preferably V) and not replaceable, the amino acid at position 309 is W and not replaceable, the amino acid at position 348 is T and not replaceable and the amino acid at position 352 is E or D (most preferably E) and not replaceable. Furthermore, the amino acid at position 233 is any of G/N/M/L/R/V/A/S/T (most preferably G) and not any other amino acid. In one embodiment, the amino acid at position 106 is isoleucine and not replaceable. This polypeptide is referred to herein as 10-1-D11_{d}.

As mentioned, the present inventors have also developed polypeptides that have a very high catalytic efficiency for an RVX type nerve agent.

Thus, according to another aspect of the present invention there is provided a polypeptide comprising an amino acid sequence of phosphotriesterase (PTE) having at least 8,000 fold the catalytic efficiency for a RVX-type nerve agent as a polypeptide which consists of the sequence as set forth in SEQ ID NO: 1, when assayed at 25° C. under identical conditions, wherein the amino acid sequence comprises the mutations K77A, A80V/M, I106A, F132E, T173N, G208D, H254G, I274N, Y309W and D233G/N/M/L/R/V/A/S/T, wherein the numbering of the mutations is according to PDB 1HZY crystal structure numbering.

In one embodiment, the PTE polypeptides have a higher catalytic efficiency for a RVX-type nerve agent than a VX-type nerve agent.

According to a specific embodiment, polypeptides which comprise improved hydrolytic efficiency of PTE to RVX-type nerve agent substrates comprise each of the mutations K77A, A80V, I106A, F132E, T173N, G208D, D233G, H254G, I274N and Y309W.

In another embodiment, the polypeptides with the above described mutations further comprise each of the following stabilizing mutations:

R118E, A203D, S222D, S238D, M293V/A, G348T and T352E/D.

PTE polypeptides which each of these mutations may have a catalytic efficiency of about 23×10⁶M⁻¹ min⁻¹ for Sp-VX and about 6.3×10⁶M⁻¹ min⁻¹ for Sp-RVX.

Thus, according to a specific embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 26 or 43 as determined using the Standard protein-protein BLAST [blastp] software of the NCBI, with the restriction that the amino acid at position 77 is A and not replaceable, the amino acid at position 80 is V or M (preferably V) and not replaceable, the amino acid at position 106 is A and not replaceable, the amino acid at position 118 is E and not replaceable, the amino acid at position 132 is E and not replaceable, that the amino acid at position 173 is N and not replaceable, the amino acid at position 203 is D and not replaceable, the amino acid at position 208 is D and not replaceable, the amino acid at position 222 is D and not replaceable, the amino acid at position 238 is D and not replaceable, the amino acid at position 254 is G and not replaceable, the amino acid at position 274 is N and not replaceable, the amino acid at position 293 is V/A (most preferably V) and not replaceable, the amino acid at position 309 is W and not replaceable, the amino acid at position 348 is T and not replaceable and the amino acid at position 352 is E or D (most preferably E) and not replaceable. Furthermore, the amino acid at position 233 is any of G/N/M/L/R/V/A/S/T (most preferably G) and not any other amino acid. This polypeptide is referred to herein as 1-3-D5^{d} I106A.

Another exemplary polypeptide according to this aspect of the present invention comprises each of the mutations C59M, K77A, A80V, I106A, F132E, T173N, G208D, D233G, H254G, A266Del, I274N and Y309W.

Polypeptides with the above described mutations may further comprise each of the following stabilizing mutations:

R118E, A203D, S222D, S238D, M293V/A, G348T and T352E/D.

PTE polypeptides which each of these mutations may have a catalytic efficiency of about 3.5×10⁶M⁻¹ min⁻¹ for Sp-VX and about 12×10⁶M⁻¹ min⁻¹ for Sp-RVX.

Thus, according to a specific embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 15 or 38 as determined using the Standard protein-protein BLAST [blastp] software of the NCBI, with the restriction that the amino acid at position 59 is M and not replaceable, the amino acid at position 77 is A and not replaceable, the amino acid at position 80 is V or M (preferably V) and not replaceable, the amino acid at position 106 is A and not replaceable, the amino acid at position 118 is E and not replaceable, the amino acid at position 132 is E and not replaceable, that the amino acid at position 173 is N and not replaceable, the amino acid at position 203 is D and not replaceable, the amino acid at position 208 is D and not replaceable, the amino acid at position 222 is D and not replaceable, the amino acid at position 238 is D and not replaceable, the amino acid at position 254 is G and not replaceable, the amino acid at position 266 is deleted, the amino acid at position 274 is N and not replaceable, the amino acid at position 293 is V/A (most preferably V) and not replaceable, the amino acid at position 309 is W and not replaceable, the amino acid at position 348 is T and not replaceable and the amino acid at position 352 is E or D (most preferably E) and not replaceable. Furthermore, the amino acid at position 233 is any of G/N/M/L/R/V/A/S/T (most preferably G) and not any other amino acid. This polypeptide is referred to herein as IVA1-m2p0^{d}.

Another exemplary polypeptide according to this aspect of the present invention is the one having each of the mutations K77A, A80M, I106A, F132E, T173N, G208D, D233G, H254G, A270S, L271W, I274N and Y309W.

This polypeptide may further comprise each of the following stabilizing mutations: R118E, A203D, S222D, S238D, M293V/A, G348T and T352E/D.

PTE polypeptides which each of these mutations may have a catalytic efficiency of about 7×10⁶M⁻¹ min⁻¹ for Sp-VX and about 8.3×10⁶M⁻¹ min⁻¹ for Sp-RVX.

Thus, according to a specific embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologus/identical to the sequence as set forth in SEQ ID NO: 28 or 44 as determined using the Standard protein-protein BLAST [blastp] software of the NCBI, with the restriction that the amino acid at position 77 is A and not replaceable, the amino acid at position 80 is V or M (preferably M) and not replaceable, the amino acid at position 106 is I and not replaceable, the amino acid at position 118 is E and not replaceable, the amino acid at position 132 is E and not replaceable, that the amino acid at position 173 is N and not replaceable, the amino acid at position 203 is D and not replaceable, the amino acid at position 208 is D and not replaceable, the amino acid at position 222 is D and not replaceable, the amino acid at position 238 is D and not replaceable, the amino acid at position 254 is G and not replaceable, the amino acid at position 270 is S and not replaceable, the amino acid at position 271 is W and not replaceable, the amino acid at position 274 is N and not replaceable, the amino acid at position 293 is V/A (most preferably V) and not replaceable, the amino acid at position 309 is W and not replaceable, the amino acid at position 348 is T and not replaceable and the amino acid at position 352 is E or D (most preferably E) and not replaceable. Furthermore, the amino acid at position 233 is any of G/N/M/L/R/V/A/S/T (most preferably G) and not any other amino acid. This polypeptide is referred to herein as 10-2-C3^{d}-I106A.

Another exemplary polypeptide is the one having each of the mutations K77A, A80M, F132E, T173N, G208D, D233G, H254G, S267M, A270S, L271W, I274N and Y309W.

This polypeptide may further comprise each of the following stabilizing mutations: R118E, A203D, S222D, S238D, M293V/A, G348T and T352E/D.

PTE polypeptides which each of these mutations may have a catalytic efficiency of about 8×10⁶M⁻¹ min⁻¹ for Sp-VX and about 10×10⁶M⁻¹ min⁻¹ for Sp-RVX.

Thus, according to a specific embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 23 or 42 as determined using the Standard protein-protein BLAST [blastp] software of the NCBI, with the restriction that the amino acid at position 77 is A and not replaceable, the amino acid at position 80 is V or M (preferably M) and not replaceable, the amino acid at position 106 is A and not replaceable, the amino acid at position 118 is E and not replaceable, the amino acid at position 132 is E and not replaceable, that the amino acid at position 173 is N and not replaceable, the amino acid at position 203 is D and not replaceable, the amino acid at position 208 is D and not replaceable, the amino acid at position 222 is D and not replaceable, the amino acid at position 238 is D and not replaceable, the amino acid at position 254 is G and not replaceable, the amino acid at position 267 is M and not replaceable, the amino acid at position 270 is S and not replaceable, the amino acid at position 271 is W and not replaceable, the amino acid at position 274 is N and not replaceable, the amino acid at position 293 is V/A (most preferably V) and not replaceable, the amino acid at position 309 is W and not replaceable, the amino acid at position 348 is T and not replaceable and the amino acid at position 352 is E or D (most preferably E) and not replaceable. Furthermore, the amino acid at position 233 is any of G/N/M/L/R/V/A/S/T (most preferably G) and not any other amino acid. This polypeptide is referred to herein as 10-1-D11^{d}-I106A.

According to another embodiment, the polypeptides of this aspect of the present invention comprise a sequence at least 99% or 100% homologous/identical to any of the sequences as set forth in SEQ ID NO: 15, 17, 19, 21, 23, 26 or 28.

In a further embodiment, the polypeptides of this aspect of the present invention consist of a sequence at least 99% or 100% homologous/identical to any of the sequences as set forth in SEQ ID NO: 15, 17, 19, 21, 23, 26 or 28.

It will be appreciated that in order to aid in isolation of the protein, the protein may be expressed with additional amino acid sequences (i.e., tags) engineered to enhance stability, production, purification, yield or toxicity of the expressed polypeptide. Such a fusion protein can be designed so that the fusion protein can be readily isolated by affinity chromatography; e.g., by immobilization on a column specific for the heterologous protein.

Examples of affinity tags include, but are not limited to HIS, CBP, CYD (covalent yet dissociable NorpD peptide), Strep II, FLAG, HPC (heavy chain of protein C) peptide tags, and the GST and MBP protein fusion tag systems.

According to a particular embodiment, the affinity tag is maltose binding protein (MBP).

According to a particular embodiment, the affinity tag is MBP and the sequence is adapted for expression in E. Coli (e.g. devoid of the first 29 amino acids of SEQ ID NO: 35).

Thus, the polypeptide may comprise any of the above described polypeptides except that the first methionine amino acid is replaced with the MBP sequence (SEQ ID NO: 29).

The affinity tag may be attached directly to the PTE sequence or via a peptide linker. An exemplary linker is set forth in SEQ ID NO: 30.

Exemplary PTE polypeptides contemplated by the present invention which include the MBP affinity tag are those that are at least 99% or 100% identical to SEQ ID NOs: 5, 7, 9, 11, 13, 25 and 27.

The term “polypeptide” as used herein encompasses native polypeptides (synthetically synthesized polypeptides or recombinant polypeptides) and peptidomimetics, as well as peptoids and semipeptoids which are peptide analogs, which may have, for example, modifications rendering the polypeptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

Synthetic amino acid substitutions may be employed to improve stability and bioavailability.

Table 1A below lists non-conventional or modified amino acids e.g., synthetic, which can be used with the present invention.

TABLE 1A

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The present teachings also provide for nucleic acid sequences encoding such PTE polypeptides.

Thus, according to an aspect of the present invention there is provided an isolated polynucleotide including a nucleic acid sequence, which encodes the isolated polypeptide of the present invention.

As used herein the phrase “an isolated polynucleotide” refers to a single or a double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Polypeptides of the present invention can be synthesized using recombinant DNA technology or solid phase technology.

Recombinant techniques are preferably used to generate the polypeptides of the present invention. Such recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

To produce a polypeptide of the present invention using recombinant technology, a polynucleotide encoding a polypeptide of the present invention is ligated into a nucleic acid expression construct, which includes the polynucleotide sequence under the transcriptional control of a cis-regulatory (e.g., promoter) sequence suitable for directing constitutive or inducible transcription in the host cells, as further described hereinbelow.

Exemplary polynucleotide sequences for expressing the polypeptides of the present invention in bacterial systems are set forth in SEQ ID NOs: 6, 8, 10, 12, 14, 16, 18, 20, 22, 24.

Other than containing the necessary elements for the transcription and translation of the inserted coding sequence, the expression construct of the present invention can also include sequences (i.e., tags) engineered to enhance stability, production, purification, yield or toxicity of the expressed polypeptide. Such a fusion protein can be designed so that the fusion protein can be readily isolated by affinity chromatography; e.g., by immobilization on a column specific for the heterologous protein. Where a cleavage site is engineered between the peptide moiety and the heterologous protein, the peptide can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

As mentioned, a variety of prokaryotic or eukaryotic cells can be used as host-expression systems to express the polypeptide coding sequence. These include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence. Mammalian expression systems can also be used to express the polypeptides of the present invention. Bacterial systems are preferably used to produce recombinant polypeptides, according to the present invention, thereby enabling a high production volume at low cost.

Other expression systems such as insects and mammalian host cell systems, which are well known in the art can also be used by the present invention.

In any case, transformed cells are cultured under effective conditions, which allow for the expression of high amounts of recombinant polypeptides. Effective culture conditions include, but are not limited to, effective media, bioreactor, temperature, pH and oxygen conditions that permit protein production. An effective medium refers to any medium in which a cell is cultured to produce the recombinant polypeptides of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

Following a certain time in culture, recovery of the recombinant protein is effected. The phrase “recovering the recombinant protein” refers to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Polypeptides of the present invention can be used for treating an organophosphate exposure associated damage.

Thus according to an aspect of the invention there is provided a method of treating or preventing organophosphate exposure associated damage in a subject in need thereof, the method comprising providing the subject with a therapeutically effective amount of at least one of the isolated polypeptides described above to thereby treat the organophosphate exposure associated damage in the subject.

The particular PTE polypeptide may be selected according to its activity. In one embodiment a PTE polypeptide may be selected which has a high catalytic activity towards both CVX and RVX. The polypeptide named IVA1-m2p0 may provide effective protection from both. In another embodiment, a PTE polypeptide may be selected which is capable of hydrolyzing the toxic isomers of GA or GB—for example 10-2-C3 or 10-1-D11). Contemplated combinations of PTE variants include 10-2-C3 together with IVA1-M2pO, 10-2-C3 together with 10-2-C3-I106A or 10-2-C3 together with 10-1-D11-I106A. Additional contemplated combinations of PTE variants include 10-1-D11 together with IVA1-M2pO, 10-1-D11 together with 10-2-C3-I106A or 10-1-D11 together with 10-1-D11-I106A.

As used herein the term “treating” refers to preventing, curing, reversing, attenuating, alleviating, minimizing, suppressing or halting the deleterious effects of the immediate life-threatening effects of organophosphate intoxication and its long-term debilitating consequences.

As used herein the phrase “organophosphate exposure associated damage” refers to short term (e.g., minutes to several hours post-exposure) and long term damage (e.g., one week up to several years post-exposure) to physiological function (e.g., motor and cognitive functions). Organophosphate exposure associated damage may be manifested by the following clinical symptoms including, but not limited to, headache, diffuse muscle cramping, weakness, excessive secretions, nausea, vomiting and diarrhea. The condition may progress to seizure, coma, paralysis, respiratory failure, delayed neuropathy, muscle weakness, tremor, convulsions, permanent brain dismorphology, social/behavioral deficits and general cholinergic crisis (which may be manifested for instance by exacerbated inflammation and low blood count. Extreme cases may lead to death of the poisoned subjects.

As used herein the term “organophosphate compound” refers to a V-type organophosphate, as described herein above.

As used herein the phrase “a subject in need thereof” refers to a human or animal subject who is sensitive to OP toxic effects. Thus, the subject may be exposed or at a risk of exposure to OP. Examples include civilians contaminated by a terrorist attack at a public event, accidental spills in industry and during transportation, field workers subjected to pesticide/insecticide OP poisoning, truckers who transport pesticides, pesticide manufacturers, dog groomers who are overexposed to flea dip, pest control workers and various domestic and custodial workers who use these compounds, military personnel exposed to nerve gases.

As mentioned, in some embodiments of the invention the method is effected by providing the subject with a therapeutically effective amount of the PTE polypeptide of the invention.

As OP can be rapidly absorbed from lungs, skin, gastro-intestinal (GI) tract and mucous membranes, PTE may be provided by various administration routes or direct application on the skin.

For example, the PTE may be immobilized on a solid support e.g., a porous support which may be a flexible sponge-like substance or like material, wherein the PTE is secured by immobilization. The support may be formed into various shapes, sizes and densities, depending on need and the shape of the mold. For example, the porous support may be formed into a typical household sponge, wipe or tissue paper.

For example, such articles may be used to clean and decontaminate wounds, while the immobilized PTE will not leach into a wound. Therefore, the sponges can be used to decontaminate civilians contaminated by a terrorist attack at a public event.

Alternatively or additionally, PTE may be administered to the subject per se or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the PTE accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, dermal, transmucosal, especially transnasal, intestinal or parenteral delivery, including intramuscular, subcutaneous and intramedullary injections as well as intrathecal, direct intraventricular, intravenous, intraperiotoneal, intranasal, intrabone or intraocular injections.

Alternately, one may administer the pharmaceutical composition in a local rather than systemic manner, for example, via injection of the pharmaceutical composition directly into a tissue region (e.g., skin) of a patient. Topical administration is also contemplated according to the present teachings.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer (see the Examples section which follows). Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

PTE may be administered prior to the OP exposure (prophylactically, e.g., 10 or 8 hours before exposure), and alternatively or additionally administered post exposure, even days after (e.g., 7 days) in a single or multiple-doses.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

The ability of PTE to sequester OP molecules, suggests use of same in the decontamination of OP contaminated surfaces and detoxification of airborne OP.

Thus, an aspect of the invention further provides for a method of detoxifying a surface contaminated with an OP molecule; or preventing contamination of the surface with OP. The method is effected by contacting the surface with PDE.

Thus, synthetic and biological surfaces contemplated according to embodiments of the invention include, but are not limited to, equipment, laboratory hardware, devices, fabrics (clothes), skin (as described above) and delicate membranes (e.g., biological). The mode of application will very much depend on the target surface. Thus, for example, the surface may be coated with foam especially when the surface comprises cracks, crevices, porous or uneven surfaces. Application of small quantities may be done with a spray-bottle equipped with an appropriate nozzle. If a large area is contaminated, an apparatus that dispenses a large quantity of foam may be utilized.

Coatings, linings, paints, adhesives sealants, waxes, sponges, wipes, fabrics which may comprise the PTE may be applied to the surface (e.g., in case of a skin surface for topical administration). Exemplary embodiments for such are provided in U.S. Pat. Application No. 20040109853.

Surface decontamination may be further assisted by contacting the surface with a caustic agent; a decontaminating foam, a combination of baking condition heat and carbon dioxide, or a combination thereof. Sensitive surfaces and equipments may require non corrosive decontaminants such as neutral aqueous solutions with active ingredient (e.g., paraoxonases).

In addition to the above described coating compositions, OP contamination may be prevented or detoxified using an article of manufacture which comprise the PTE immobilized to a solid support in the form of a sponge (as described above), a wipe, a fabric and a filter (for the decontamination of airborne particles). Chemistries for immobilization are provided in U.S. Pat. Application 20040005681, which is hereby incorporated in its entirety.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

PTE Variant Library Construction:

The PTE gene was cloned using EcoRI and PstI sites into a pMALc2x expression vector (NEB®) fused to an N-terminal maltose binding protein (MBP) tag. Libraries of PTE variants were constructed using the methods described in Table 2 and below:

TABLE 2
Summary of library making strategies

| Strategy | Description |
|---|---|
| I | Amino acid substitutions (typically ≥3 per site), targeted to one |
| | or more sites, applied to either single or multiple variants |
| | without shuffling. |
| | Using on average ~2 substitutions per clone. |
| II | Shuffling of selected variants with or without the incorporation |
| | of new mutations. |
| III | Simultaneous mutagenesis of sets of positions (typically 4-6 per |
| | variant) using site-specific substitutions. |
| IV | Rationally designed, site directed mutants with up to 3 mutations |
| | per variant. |
| V | Whole gene random mutagenesis |

1. Site Directed Mutagenesis Using Single Point Mutations or Degenerate Codons (Table 2, I, III, IV).

Single point mutations or degenerate NNK codons were encoded in the center of 22-28 bp synthetic oligonucleotides (oligo's). A reverse oligo encoding the complementary region was used for whole-plasmid PCR amplification using PfuUltra™ II Fusion HS DNA Polymerase. The original plasmid was eliminated by digestion with DpnI (NEB™). The PCR product was purified using a PCR purification kit (QIAGEN™), phosphorylated using T4 PNK (NEB™) and 10 mM ATP (SIGMA) and ligated using T4 DNA ligase (Thermo™). Ligation products were purified by ethanol precipitation and transformed to electro-competent E.cloni™ cells (Lucigen®). Cells were plated on LB-agar plates with Amp (100 mg/1). Plasmids containing the correct sequence were identified by colony PCR using Taq polymerase (Bio-ReadyMix™), isolated and transformed into the expression strain GG48 (Grass et al., 2001) for screening.

Generating Targeted Libraries by the Combinatorial Incorporation of Synthetic Oligonucleotides During Gene Shuffling (Table 2, II).

Briefly, amino-acid substitutions were introduced using short oligonucleotides encoding the mutated site flanked by the wild-type sequences. These were added to mixtures of 50-250 bp fragments of selected PTE variants, PCR amplified and cloned into a pMAL-c2x expression vector using EcoRI and PstI sites as described in (Rockah-Shmuel et al., 2014). The protocol was tuned to obtain a combinatorial incorporation of the designed mutations at an average of 2±1 mutations per gene.

Shuffled Clones Libraries (Table 2, II).

Selected clones were PCR amplified using iPFU ready-mix (Intron™), DpnI digested and purified as described above. They were mixed at equal concentrations to a total of 20 μg DNA mix, digested to 50-250 bp, purified and assembled to whole genes by PCR as described (Rockah-Shmuel, et al., 2014) without the introduction of spiking oligos. They were then cloned into the expression vector as described above.

Random Mutagenesis Libraries (Table 2, V).

Whole-gene random mutagenesis was performed using the GeneMorph™ II Random Mutagenesis Kit (Stratagene™) and oligos immediately upstream and downstream to MBP-PTE's ORF, using only 10 cycles and 0.5 μg template according to the manufacturer's protocol. The PCR product was then digested with DpnI, purified on Micro Bio-Spin™ 6 Columns (BioRad™), and amplified by PCR using iPFU ready-mix (Intron™). The resulting gene library was digested with EcoRI-HF™ and PstI-HF™ and cloned into a pMALc2x (NEB™) vector.

Computational Library Design:

Initially, a transition state (TS) model was constructed with an in-line nucleophilic attack by a hydroxide on the phosphorus atom idealizing the TS geometry to a trigonal bipyramidal geometry (Aubert et al., 2004). The TS models were superimposed onto the binuclear metal site of PTE variant C23 using the crystal structures with the phosphoryl oxygen coordinating the Zn(3 atom and using the RossettaDock software suite (Davis and Baker, 2009, Meiler and Baker, 2006) to explore energetically favorable alignments. The top scoring docked poses were visually inspected and chosen for design and rigid body minimization. A distance restraint of 2.15+/−0.25 Å was added between the phosphoryl oxygen and Zn(3 atom and a rigid-body minimization was performed using Talaris2014 (Leaver-Fay et al., 2013) with RosettaScripts (Fleishman et al., 2011). Interactions between models and enzyme were optimized using RosettaDesign algorithm (Kuhlman et al., 2003). Substitutions were evaluated using a position-specific scoring matrix computed using psipred (McGuffin et al., 2000) based on the phosphotriesterase sequence from Brevundimonas diminuta followed by AAG calculations using the AAG monomer application (Kellogg et al., 2011).

Computational Design of Stabilized C23 Variants:

To design stable protein variants of C23 the PROSS algorithm (Goldenzweig, et al., 2016) was applied on wild type PTE, (pdb entry: 1HZY, chain A), and the minimal sequence identity threshold was lowered to 28% (default is 30%) to increase the diversity of homologs. To preserve PTE function, 39 residues at its active site region (defined as residue numbers in pdb entry 1HZY that match the equivalent residues in pdb entry 4NP7 that are within 8 Å from DPJ) and 35 residues at PTE's dimeric interface (namely residues within 5 Å, from chain B) were held fixed during all simulation steps. Three designs were selected for experimental testing; DNA sequences bearing the stabilizing mutations, the C23 mutations and codon optimized for E. coli expression were ordered as synthetic genes from Gen9. The genes were amplified by PCR using external primers and cloned into the expression vector pMALc2x using EcoRI and PstI restriction sites.

PTE Library Screening:

Screening was similar to the previously described procedure (Cherny, et al., 2013). Briefly, randomly picked colonies were individually grown O/N in 96-Deep-Well plates (Axygen). Overnight cultures were used to inoculate (1:100 dilution) 0.5 ml LB medium with 100 μg/ml ampicillin and 0.1 mM ZnCl₂, grown to OD⁶⁰⁰ nm≈0.6, induced with 0.4 mM IPTG, and grown at R.T O/N. Cells were pelleted and lysed by resuspension and shaking in 300 al/well of lysis buffer (0.1 M Tris pH 8.0, 0.1 M NaCl, 0.1% v/v Triton-X100, 0.2 mg/ml lysozyme, 10 mM Na₂CO₃ and 1:50,000 Benzonase nuclease). Lysates were centrifuged and kept at 4° C. O/N before screening. 40 μl of clear cell lysate from each well were mixed with 20 μl of in situ generated V-agents (50-4000 nM) and 40 μl of pure hAChE (human acetylcholinesterase, 2.5 nM). The reaction mixtures were incubated for 60 minutes before determination of residual AChE activity by mixing 20 μl of the reaction mixture with 180 μl PBS containing 0.85 mM DTNB and 0.55 mM acetylthiocholine. Initial velocities of acetylthiocholine hydrolysis were determined at 412 nm using a Powerwave HT spectrophotometer (BioTek).

Enzyme Expression and Purification:

Was performed as previously described (Cherny, et al., 2013). The recombinant PTE variants (MBP N-terminal fusion) were purified as follows: The gene was cloned into a pMALc2x expression vector (NEB™) and transformed into E. coli BL21/DE3 cells. The culture grew in 2YT medium including ampicillin overnight at 37° C. The inoculate was dilute 1:100 into LB medium with ampicillin (100 μg/ml) and 0.2 mM ZnCl₂ and grown at 37° C. to OD₆₀₀ nm≈0.6. IPTG was added (0.4 mM), and the culture was allowed to grow overnight at 20° C. Cells were harvested by centrifugation and re-suspended in Lysis buffer (0.1 M Tris pH 8.0, 0.1 M NaCl, 10 mM NaHCO₃, 0.1 mM ZnCl₂, 0.4 mg/ml Lysozyme (Sigma), 1:500 diluted protease inhibitor cocktail (Sigma), 50 Units Benzonase nuclease (Merk)). Cells were then lysed using sonication, clarified by centrifugation (10,000×g, 4° C., 30 min) and passed through a column packed with amylose beads pre-equilibrated with ‘wash buffer’ (0.1 M Tris pH 8.0, 0.125 M NaCl, 10 mM NaHCO₃, 0.1 mM ZnCl₂). Following an extensive (10 c.v) wash using the ‘wash buffer’, the MBP-PTE fusion proteins were eluted with ‘wash buffer’ supplemented with 10 mM maltose. The fractions containing pure MBP-PTE variants were pooled and dialyzed over night at 4° C. with 0.1 M Tris pH 8.0, 0.1 M NaCl. Protein concentrations were examined by absorbance at 280 nm (extinction coefficient value for the MBP fused enzymes was ε=95925 OD/M) and by PAGE-gel densitometry using BSA standards for calibration.

Human AChE was cloned into the pHLsec expression vector (Aricescu et al., 2006) and produced in large scale in HEK293T cells. The secreted protein was purified from the medium by affinity chromatography (Sussman et al., 1988).

Enzyme Kinetics: Determining the Hydrolysis Rates of Individual V-Type Nerve Agent Isomers or Racemic Mixtures Using the DTNB Assay.

Was performed as previously described (Cherny, et al., 2013). Hydrolysis rates of the individual isomers of VX or RVX and of racemic Chinese VX (CVX) were monitored by following the release of their thiol leaving groups using DTNB (Ellman et al., 1961). To a cuvette containing a 1 ml solution of 50 mM Tris-HCl (pH 8.0), 50 mM NaCl, and 0.7 mM DTNB (Ellman's reagent), we added purified PTE variants (final conc' range of 0.01-0.2 μM) and the mixture was pre-incubated at 25° C. for 4 min. The hydrolysis reaction was initiated by the addition of V-agents (final concentration range of 10-30 μM). The increase in the concentration of thio-(2-nitro)-benzoic acid (Ellman's chromophore) in the reaction mixture as function of the reaction time was monitored continuously by measuring absorbance at 412 nm. Measurements were continued until ≥95% of the expected maximal absorbance was reached. The absorbance relating to 100% hydrolysis of the examined V-agent was determined separately by replacing the PTE solution with a solution of 0.5 M NaF in 50 mM phosphate buffer, pH 8.0. Data points were fitted to a mono exponential equation and the apparent first order rate constant was divided by the PTE concentration to obtain k_{cat}/K_{M}.

Enzyme Kinetics: Monitoring the Detoxification Rates of Nerve Agents Using the AChE Assay.

Was similar to the procedure previously described (Cherny, et al., 2013). Purified PTE variants (2.5-20 μl) were added to a buffered solution (1 ml, 50 mM Tris-HCl pH 8.0, 50 mM NaCl) containing 0.2-0.5 μM of the nerve-agent substrates. The final PTE concentration range was 1 to 200 nM. Solutions of individual nerve agent isomers (10-50 μM) were generated in water (details available upon request). At selected time intervals, aliquots (5-10 μl) were removed from the hydrolysis reaction into solutions of recombinant human acetylcholinesterase (0.2-0.5 ml, 10 mM phosphate buffer pH 8.0, final hAChE conc' 4-7 nM). The dilute aliquots were incubated for 60 min at RT to ensure complete interaction between the OPs and hAChE. Maximal hAChE inhibition was determined by dilution of the same OP concentration into the buffer solution (50 mM Tris-HCl pH 8.0, 50 mM NaCl) in the absence of the PTE variant, and kept at ≤90% by adjusting the OP concentration accordingly. Residual hAChE activity was determined by diluting the aliquot solution (50-100 fold) into a solution of acetylthiocholine iodide (ATC) (1 mM) and DTNB (0.7 mM) and monitoring absorbance at 412 nm over 1.5 min. The hAChE activity assay was repeated for each sample after 75-80 min to ascertain the percentage of hAChE inhibition. The spontaneous hydrolysis of ATC was subtracted from all ATC hydrolysis rates. The spontaneous hydrolysis of the nerve agents in 50 mM Tris, 50 mM NaCl pH 8.0 was negligible over the time period used to monitor detoxification by the PTE variants. The percentage of hAChE inhibition was calculated from the ratio of residual hAChE activity at each time point to the residual hAChE activity at t=0 (before the addition of PTE). The apparent first-order detoxification rate constant (k) was obtained by fitting a mono exponential equation to the plot of the % hAChE inhibition as a function of the incubation time with PTE. The catalytic efficiency k_{cat}/K_{M} was obtained by dividing k by the molar concentration of PTE in the solution.

Thermal Inactivation Assay:

Single colonies of GG48 cells expressing PTE variants were picked from agar plates, grown in replicates in 96 deep-well plates, induced, pelleted and lysed as described above (PTE library screening). Samples of clarified cell lysates (20 al) were transferred to a 96-well PCR plate using a liquid handling robot Precision 2000. The PCR plate was sealed with a PCR Polyethylene 96 Well Microplate Sealing Tape. The samples were incubated at different temperatures (45-70° C.) in a thermal cycler gradient PCR for 30 min and cooled for 10 min at 4° C. Paraoxonase activity assay: The samples were dilute 1:200 in PTE activity buffer without zinc (0.1 M Tris pH 8.0, 0.1 M NaCl) and 5 al samples were taken to a 96-well, flat bottom, ELISA plate and mixed with 195 al of a freshly made solution of paraoxon 0.2 mM in PTE activity buffer with no zinc. The OD^{405 nm} of each well was recorded for ≥5 min in a BioTek Synergy HT ELISA reader. Duplicates of each variant at each temperature were assayed. Residual room-temperature activity was calculated by comparing the initial velocities of paraoxon hydrolysis for each variant at each temperature with similar assay results for samples kept at room temperature. The T_{1/2} inactivation temperature was calculated as the temperature leading to a residual room temperature activity of 50%.

Metal Chelation Assay:

Samples of clarified cell lysates, following the expression of PTE variants, were prepared and dispensed in 20 al volumes to 96-well PCR plates as described above (see Thermal inactivation assay). To each well containing the lysate samples, a solution of 20 al of 1,10-phenanthrolin (final conc' 50 μM)—using the precision 2000 robot (BioTek™) was added. The plate was then incubated for 30 min at 37° C., and cooled to room temp. Incubated samples were diluted 1:200 in 0.1 M Tris pH 8.0, 0.1 M NaCl. Samples of the dilute solutions (5 al) were taken to 96-well ELISA plates for measuring paraoxonase activity using 195 al Paraoxon 0.2 mM in 0.1 M Tris pH 8.0, 0.1 M NaCl as described above. The residual paraoxonase activity was calculated using similarly treated control samples that were incubated with buffer 0.1 M Tris pH 8.0, 0.1 M NaCl instead of phenanthrolin. Each variant was measured in 24 replicates.

### Example 1

### Optimization of V-Agent Hydrolyzing PTE Variants

The starting point PTE variant in this work is named C23 (SEQ ID NO: 31). This was the end point of the directed evolution effort to improve the efficiency of V-type nerve agent hydrolysis by PTE disclosed in Cherny, et al., 2013. Its catalytic efficiency was 5×10⁶ M⁻¹ min⁻¹ with the toxic Sₚ isomer of VX, and 3.4×10⁶ M⁻¹ min⁻¹ with the Sₚ isomer of RVX (Table 3, herein below). C23 was the outcome of five rounds of directed evolution, whereby a round consisted of the following steps: generation of a gene library from the best variants of the previous round; a screen for variants with higher detoxifying rates; isolation and verification of improved variants; and finally, the purification and determination of catalytic efficiencies of these variants.

TABLE 3
Catalytic efficiencies of hydrolysis of Sₚ-VX and Sₚ-RVX.
(k_{cat}/K_{M}) × 10⁶M⁻¹ min⁻¹ ± SD

| Round # | Variant | Mutational Composition^{a} | Sₚ-VX^{b} | Sₚ-RVX^{b} |
|---|---|---|---|---|
| 0 | PTE-S5^{c} | — | 0.01 ± 0.003 | 7 × 10⁻⁴ ± 1 × 10⁻⁵ |
| | | | (1) | (1) |
| 5 | C23^{c} | K77A, A80V, F132E, T173N, | 5 ± 0.5 | 0.7 ± 0.02 |
| | | G208D, H254G, I274N | (500) | (1000) |
| 10 | C23- | K77A, A80V, F132E, T173N, | 7 ± 0.3 | 0.8 ± 0.02 |
| | Y309W | G208D, H254G, I274N, Y309W | (700) | (1143) |
| 11 | C23- | K77A, A80V, F132E, T173N, | 15 ± 1.0 | 1.4 ± 0.20 |
| | Y309W- | G208D, H254G, I274N, Y309W | (1500) | (2000) |
| | m2p0^{d} | | | |
| 11 | 4E11^{d} | K77A, A80V, F132E, T173N, | 31 ± 3.3 | 0.4 ± 0.03 |
| | | G208D, H254G, S267M, A270S, | (3100) | (571) |
| | | L271W, I274N, Y309W | | |
| 12 | IVA1- | C59M, K77A, A80V, I106A, | 3.5 ± 0.8 | 12 ± 1.30 |
| | m2p0^{d} | F132E, T173N, G208D, D233G, | (350) | (17143) |
| | | H254G, A266Del, I274N, Y309W | | |
| 13 | 1-3-D5^{d} | K77A, A80V, F132E, T173N, | 28 ± 3.0 | 2.5 ± 0.20 |
| | | G208D, D233G, H254G, I274N, | (2800) | (3571) |
| | | Y309W | | |
| ^{e} | 1-3-D5^{d} | K77A, A80V, I106C, F132E, | 6 ± 0.7 | 1.4 ± 0.50 |
| | I106C | T173N, G208D, D233G, H254G, | (600) | (2000) |
| | | I274N, Y309W | | |
| ^{e} | 1-3-D5^{d} | K77A, A80V, I106A, F132E, | 23 ± 0.1 | 6.3 ± 0.80 |
| | I106A | T173N, G208D, D233G, H254G, | (2300) | (9000) |
| | | I274N, Y309W | | |
| 13 | 10-2-C3^{d} | K77A, A80M, F132E, T173N, | 50 ± 5.0 | 3.2 ± 0.01 |
| | | G208D, D233G, H254G, A270S, | (5000) | (4571) |
| | | L271W, I274N, Y309W | | |
| ^{e} | 10-2-C3^{d}- | K77A, A80M, I106C, F132E, | 7 ± 1.0 | 1.2 ± 0.10 |
| | I106C | T173N, G208D, D233G, H254G, | (700) | (1714) |
| | | A270S, L271W, I274N, Y309W | | |
| ^{e} | 10-2-C3^{d}- | K77A, A80M, I106A, F132E, | 7 ± 0.2 | 8.3 ± 1.10 |
| | I106A | T173N, G208D, D233G, H254G, | (700) | (11857) |
| | | A270S, L271W, I274N, Y309W | | |
| 13 | 10-1-D11^{d} | K77A, A80M, F132E, T173N, | 51 ± 8.0 | 2.7 ± 1.10 |
| | | G208D, D233G, H254G, S267M, | (5100) | (3857) |
| | | A270S, L271W, I274N, Y309W | | |
| ^{e} | 10-1-D11^{d}- | K77A, A80M, I106C, F132E, | 11.5 ± 0.1 | 1.5 ± 0.20 |
| | I106C | T173N, G208D, D233G, H254G, | (1150) | (2143) |
| | | S267M, A270S, L271W, I274N, | | |
| | | Y309W | | |
| ^{e} | 10-1-D11^{d}- | K77A, A80M, I106A, F132E, | 8 ± 0.4 | 10 ± 2.40 |
| | I106A | T173N, G208D, D233G, H254G, | (800) | (14286) |
| | | S267M, A270S, L271W, I274N, | | |
| | | Y309W | | |

^{a}Substitutions relative to wt-like PTE S5 (Roodveldt and Tawfik, 2005). Substitution of Ile106 are highlighted in bold. Stabilizing mutations listed in comment (d).
^{b}In brackets - fold improvement relative to wt. like PTE S5.
^{c}Taken from (Cherny, et al., 2013).
^{d}Including stabilizing mutations: R118E, A203D, S222D, S238D, M293V, G348T and T352E.
^{e}A rationally constructed variant not obtained from a library screen.

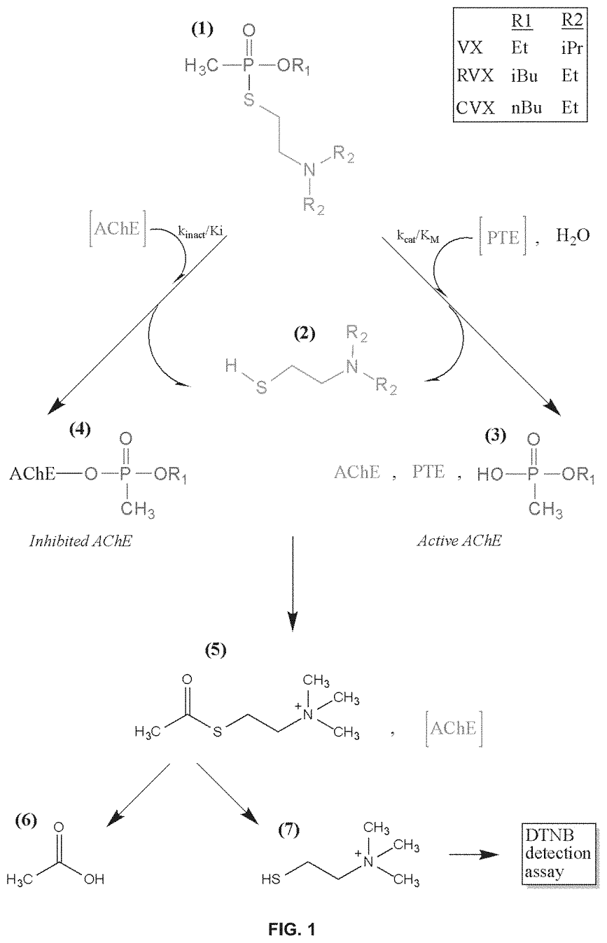

In this work, the goal was to further increase the catalytic efficiencies of C23 with V-type nerve agents. The in vitro assay that was previously developed (Goldsmith, et al., 2012) was used to measure the ability of the screened enzyme variants to prevent the inhibition of acetylcholinesterase (AChE) by organophosphates (OPs) such as VX or RVX (FIG. 1). Inactive PTE variants resulted in complete inhibition of AChE activity, as expected, while sufficiently active variants could hydrolyze some, or all, of the nerve agent before it inhibited AChE. Thus, the level of residual, uninhibited AChE activity correlated with the efficiency of the detoxifying PTE variant. The V-agents were synthesized in situ as racemates (at non-hazardous concentrations and amounts). However, since the S_{P} isomers of both VX and RVX inactivate hAChE at rates that are ˜100-fold greater than those of the R_{P} isomers (Ordentlich et al., 2004), the hydrolysis of the S_{P} isomer was the one primarily assayed.

Specifically, in each round, one, or more gene libraries of PTE were generated and cloned into an expression vector, transformed to E. coli cells, and grown on agar plates. Individual colonies were randomly picked, grown, and expressed in 96-deep well plates. The cultured cells were collected, lysed, and purified human AChE was added followed by VX, RVX (in situ prepared, at final concentration of 10-800 nM). Following a period of incubation, the residual AChE activity of the reaction mixture was measured (FIG. 1). The molar ratio of AChE to V-agents ranged from 1:10 up to 1:800, allowing the present inventors to assay for variants with increasingly higher detoxification rates. The concentrations of expressed PTE variants in the lysate were estimated to be in the same range as the AChE.

Following each round, clones that exhibited ≥2 fold residual AChE activity relative to the best variants from the previous round were isolated and retested. In this verification screen, 3-6 replicates of the initially isolated variants were grown and re-screened for AChE protection in order to eliminate false positives. Reproducibly improved clones were then purified and their catalytic efficiencies with VX and RVX were determined. The best variants from each round were then used as the starting point for the generation of the successive gene library.

Initially, 900 randomly selected variants were screened from the library of round #5 (Cherny, et al., 2013) using either VX or RVX, in order to identify additional active variants. Variants improved with RVX but not with VX were identified. The genes encoding the eight most improved RVX clones were shuffled to generate library 6-1. In parallel, library 6-2 was designed, in which two active-site residues (i.e. positions 270, 273; amino acid numbering follows PDB 1HZY), were selectively substituted on the background of C23 and two other variants described by Cherny et al. (A53 and G23). These positions were chosen based on a docking model of the transition state structure of VX and the crystal structure of variant C23. Round 6 libraries were screened using a mixture of VX and RVX in order to select for broad-spectrum variants that could efficiently hydrolyze both substrates. The 6 best variants of both libraries were purified and showed improvement of ˜2 fold yet only with RVX.

Library 7-1 was designed in order to examine substitutions at position 271 only, while library 7-2 introduced rationally and Rosetta-designed mutations. Library 7-2 was generated on the background of a shuffled mixture of the genes encoding the best variants from round 6. Following the failure of the previous rounds to identify variants with improved VX detoxification rates, the libraries were separately screened with VX and RVX. However, no improved clones were found after screening library 7-1 with VX or RVX, and initially isolated clones from library 7-2 were later found to have only minor improvements.

Round 8 explored substitutions in positions in which beneficial mutations occurred in the earliest rounds leading to variant C23. In previous work (Cherny, et al., 2013), two key beneficial mutations of C23, H254G and F132E, were selected. Position 254 was highly mutable already in the first round and the mutation H254G was introduced and selected in the second round. The mutation F132E was introduced following computational modeling for improved substrate binding using Rosetta, and was also selected in the second round. Such key adaptive mutations often interact with negative epistasis; i.e., they may be beneficial on their own but deleterious when combined with other mutations (Dellus-Gur et al., 2015). Hence, shifting evolutionary trajectories may demand, for example, reversion to wild-type sequence in one adaptive position in order to enable a second adaptive mutation to open a new trajectory (Salverda et al., 2011). To explore such options, round 8 (library 8-3) targeted substitutions to position 132 including reversion to the wild type Phe and library 8-4 targeted position 254, including reversion to Asn, a previously beneficial mutation. However, none of these substitutions improved activity. In fact, the site-specific substitution of E132D, a previously beneficial mutation, resulted in a nearly complete loss of VX hydrolysis activity, indicating that E132 not only plays a key role in evolved variants, but is also epistatic with other positions. Whilst it is not possible to exclude the existence of alternative trajectories, these results suggest that if such trajectory(s) exist they differ fundamentally from the trajectory that had been followed.

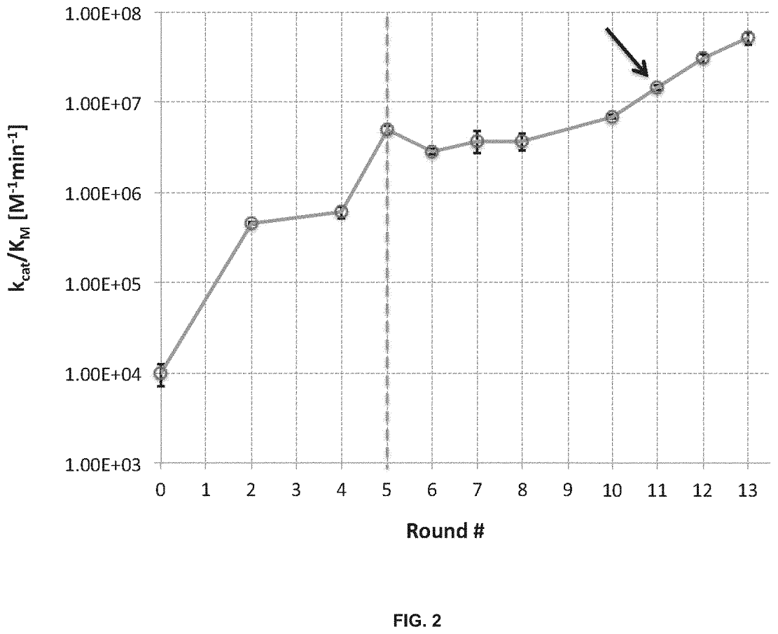

The successive rounds, 9 to 10-2, also failed to yield improved variants both in rationally and computationally designed libraries. Small improvements (˜2 fold in VX) were only observed in individually constructed variants that carried rationally designed single point mutations. At this point, it was concluded that a plateau in the improvement of catalytic efficiencies had been reached (rounds 5-10, FIG. 2).

### Example 2

### Stabilization of the Evolving PTE Variants

It was suspected that the accumulation of mutations (i.e. 9-12 per gene) in selected variants along 10 directed evolution rounds had considerably reduced their stability. This caused nearly every additional mutation to become severely destabilizing and resulted in reduced levels of active enzyme (Sikosek and Chan, 2014, Tokuriki and Tawfik, 2009). Thus, it became clear that under these conditions, library screens might fail to identify mutations that do confer improvements, even if small.

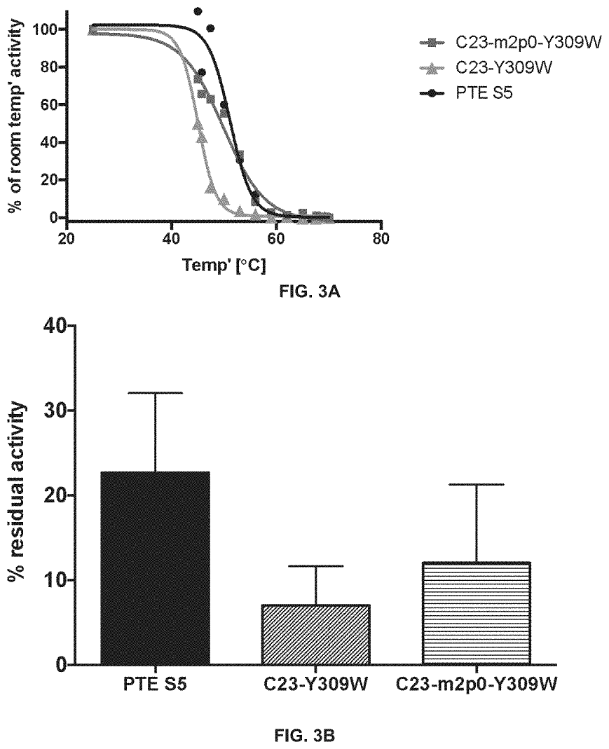

Enzyme instability is indicated, many times, by reduced expression levels of soluble, active enzyme. Variations in expression levels between variants were observed in crude cell lysates (data not shown), however, the differences were relatively small and not always correlated with specific activity. This can be attributed, most probably, to the fact that the PTE variants were expressed in fusion with a maltose binding protein (MBP, tagged at the N-terminus). MBP is known to exhibit chaperon-like properties that limit misfolding and aggregation of proteins (Kapust and Waugh, 1999). It is therefore conceivable that some mutants expressed in a soluble form, yet were enzymatically impaired. This hypothesis was later supported by the higher specific activity of the stabilized variant, as discussed below. Indeed, the inactivation mid-point temperature (i.e. the temperature at which 50% of the enzymatic activity is lost) of the best variant from round 10, C23-Y309W, which had incorporated 9 mutations (45° C.), was six degrees lower than that of the starting point PTE-S5 variant (51° C., FIGS. 3A-B).

To compensate for the loss of stability and enable further improvement in catalytic efficiency, we subjected PTE to a protein stabilization algorithm, PROSS, which designs stabilizing mutations and increases soluble protein expression without impairing protein function (Goldenzweig et al., 2016). Briefly, PROSS's workflow comprises three stages: First, it analyzes homologous sequences of the target protein and lists, for each position, a set of amino acids that appear frequently across the natural diversity of the protein family. Second, starting from a molecular structure of the target protein or of a close homologue, Rosetta computational design simulations (Whitehead et al., 2012) identify a subset of individual mutations from the above amino acid lists predicted to be independently stabilizing compared to the wild type sequence. Lastly, Rosetta's combinatorial sequence design is used to scan all combinations of mutations from the above subset to identify optimal sequence compositions, typically comprising >7 mutations each, with substantially improved native-state energy. Along this workflow, amino acid positions at functional sites are held fixed to preserve function. To design C23 for higher stability the present inventors used the crystal structure of wild-type PTE as input (pdb entry: 1HZY). Since the number of database sequences of PTE homologs is relatively low, the minimal sequence identity threshold was lowered to include homologous sequences with ≥28% similarity. Amino acid positions at the active site and at PTE's dimeric interface were held fixed. Three designs bearing 7, 16, and 25 stabilizing mutations relative to C23 (and 9, 19 and 28 relative to PTE) were selected for experimental testing.

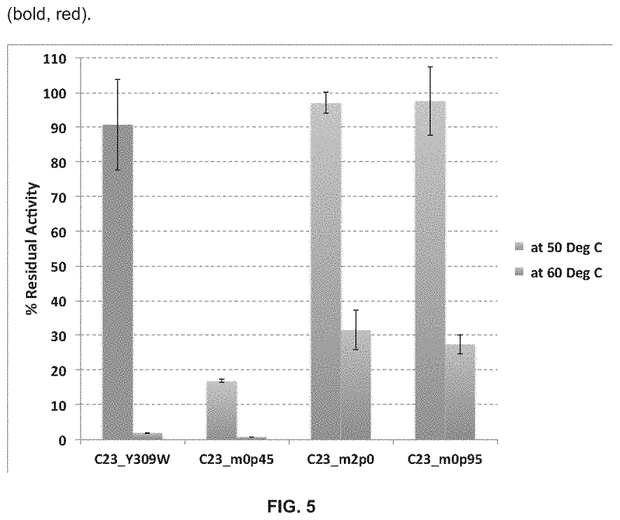

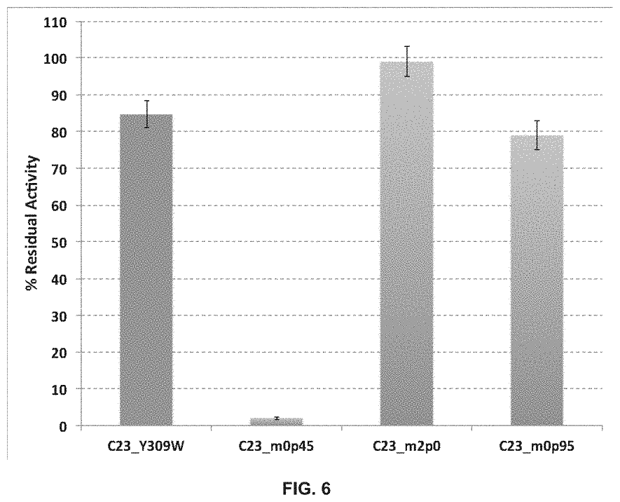

After testing these designs (FIGS. 5 and 6), the best one was chosen, m2p0, which contained 7 stabilizing mutations in addition to those of C23-Y309W and improved both in metal binding affinity and thermal stability (FIGS. 3A-B). Remarkably, the inactivation mid-point temperature of this variant was found to be 50° C. (FIGS. 3A-B), thus restoring wild-type stability to evolved variant C23-Y309W. The activity of the stabilized variant (C23-Y309W-m2p0) with VX increased by 2 fold (Table 3), although the stabilizing mutations were far from the protein's active site. This suggests that preparations of C23-Y309W contained soluble yet inactive, or poorly active enzyme.

### Example 3

### Further Optimization of Catalytic Efficiency

In the next round, 11, the present inventors explored simultaneous substitutions of positions 267, 270 and 271. The best variant from this round, 4E11, had three novel mutations and was improved by 2 fold compared to C23-Y309W-m2p0 (k_{cat}/K_{M}=3.1±0.3×10⁷ M⁻¹ min⁻¹ with Sₚ-VX, Table 2). Since the activity of this variant with VX was only 1.6 fold lower than our target goal, they turned their attention in the next round, 12, to evolve RVX hydrolyzing variants. After an improved RVX variant was identified with a catalytic efficiency of ˜1×10⁷ M⁻¹ min⁻¹ with Sₚ-RVX in round 12, the present inventors continued to improve VX hydrolyzing activity in round 13.

Along the six rounds of directed evolution for VX hydrolysis described so far and the five rounds previously described (Cherny, et al., 2013), targeted substitutions were explored at most of PTE's active-site positions. In some cases, the present inventors repeatedly explored the same positions in different rounds. Overall, this strategy yielded significant improvements, but at this stage, it seemed to have been exhausted. Thus, the present inventors decided to use whole-gene random mutagenesis to find new beneficial mutations. After screening ˜1600 clones from a random mutagenesis library of stabilized variant C23-Y309W-m2p0 (round 13-1), two beneficial mutations (V80M, D233N) were identified that appeared in several selected clones. The first one, V80M, is probably a stabilizing mutation as it occurred in a position, far from the active site, who's substitution was previously identified to improve stability (Cherny, et al., 2013, Tokuriki, et al., 2012). However, D233N is an active site mutation that was actually observed as beneficial in selected variants from earlier rounds. These two mutations were then shuffled with the mutations of variant 4E11 and with some rationally designed mutations. After screening the resulting library several variants were identified that exhibited the desired catalytic efficiency towards VX (k_{cat}/K_{M}=5×10⁷ M⁻¹ min⁻¹ with Sp-VX; Table 2). All selected variants now contained the D233G mutation.

### Example 4

### Engineering RVX Hydrolyzing Variants

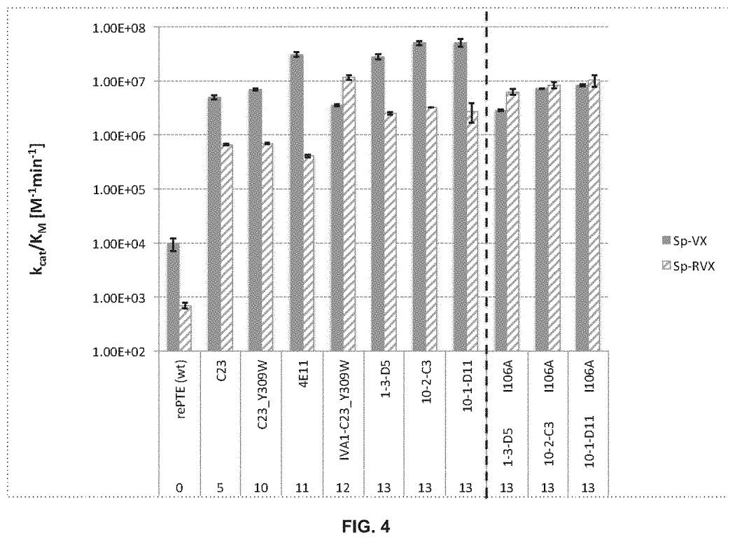

In addition to evolving efficient VX hydrolyzing variants, the present inventors wanted to evolve variants that would efficiently detoxify the threat agent RVX. The best previously evolved RVX hydrolyzing variant, A53 (Cherny, et al., 2013), exhibited 5-fold higher catalytic efficiency for S_{P}-RVX hydrolysis than C23. However, its k_{cat}/K_{M} value (3.5×10⁶ M⁻¹ min⁻¹) was >10-fold lower than the present catalytic efficiency goal, and it was also unstable with a high tendency to misfold and aggregate. Initially, the present inventors attempted to evolve C23 both for VX and RVX hydrolysis. Accordingly, in rounds 5, 7 and 8 they screened with either VX or RVX and selected only variants that showed activity on both substrates, and in round 6 they screened for detoxification of a mixture of both agents. The failure to identify improvements suggested a tradeoff between these two substrates, and this tradeoff became evident when individual variants were characterized. For example, the catalytic efficiency of the previous best round 11 variant, 4E11, with RVX was ˜78 fold lower than it efficiency with VX (FIG. 4) and 125 fold lower than the present goal (Table 2). However, an analysis of selected variants from rounds 1-7 indicated an efficient RVX hydrolyzing variant, IVA1, which had a catalytic efficiency of 5.3×10⁶ M⁻¹ min⁻¹ with Sₚ-RVX (Goldsmith et al., 2015) and did not exhibit low stability as A53.

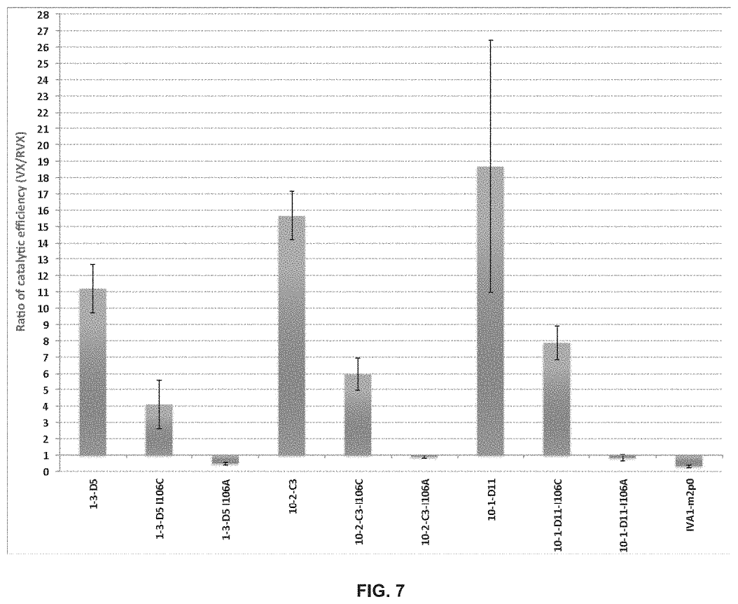

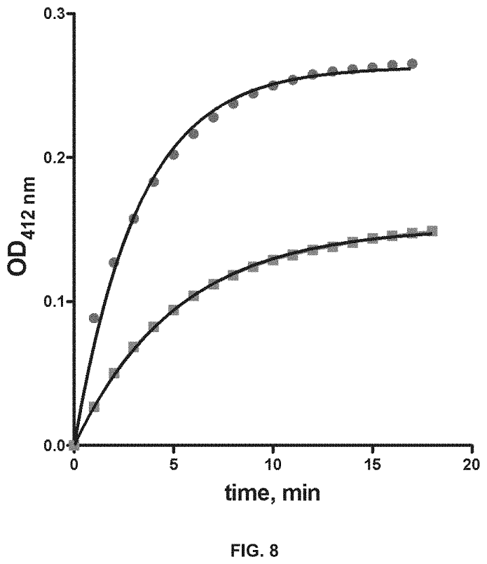

In an attempt to increase the stability of this variant for further rounds of directed evolution, the present inventors grafted the mutations of IVA1 onto the stabilized design of C23-Y309W-m2p0, and assayed the activities of the resulting IVA1-m2p0 variant (Table 2). As with C23-Y309W, stabilization had also improved the catalytic efficiency of this variant by ˜2 fold, to give a k_{cat}/K_{M} of 1.16×10⁷ M⁻¹ min⁻¹ with Sₚ-RVX, only ˜4-fold lower than the present goal. It is interesting to note that despite the fact that C23-Y309W and IVA1 differed in four positions (59, 106, 233 and 266), including a complete deletion of position 266 in IVA1, and that both differed from wt PTE S5 in 8 and 12 positions respectively, the stabilizing mutations designed using PTE S5 were able to stabilize and increase the catalytic efficiency of both variants. As previously noted (Cherny, et al., 2013, Goldsmith, et al., 2015), all variants that contained the I106A substitution exhibited more efficient hydrolysis of RVX than VX, and vice versa. Indeed, according to the present computational model, position 106 is in direct contact with the O-alkyl groups of V-type agents. The present inventors thus examined the effect of mutations of Ile106 to Ala or Cys in the three most active VX hydrolyzing variants from the last round (round 13). As can be seen from Table 2 and FIG. 7, mutating Ile106 to Cys decreased the rates with both RVX and VX. In contrast, mutating to Ala increased RVX activity by ˜3 fold while decreasing the VX rates by ˜7.5-fold. Since the initial VX/RVX activities ratio was on average 15 (FIG. 7), the mutation of Ile106 to Ala resulted in variants that have almost equal detoxification efficiencies with VX and RVX (˜1×10⁷ M⁻¹ min⁻¹, FIG. 8).

### Example 5

### Evolved Variants are Broad-Spectrum Nerve Agent Detoxifiers

The present inventors looked for hydrolases which also had broad-spectrum detoxification activity—specifically those that could hydrolyse the less toxic Rₚ isomers of V-agents, the less abundant threat agent CVX (Chinese VX) and G-type nerve agents. The large differences in their physicochemical properties make it unlikely that a single variant would efficiently hydrolyze all G-type and V-type nerve agents and their different isomers. A more likely scenario was to evolve a few variants that only differ by a few mutations and that could be combined to provide broad-spectrum prophylaxis. They thus examined the activity of their evolved variants with all these nerve-agents and their isomers (Table 4, herein below).

TABLE 4
Catalytic efficiencies of hydrolysis of nerve agents
k_{cat}/K_{M} (×10⁶ M⁻¹ min⁻¹ ± SD)

| Round | Variant | | | | | CVX- | | CVX- | | |
|---|---|---|---|---|---|---|---|---|---|---|
| # | name | Rₚ-VX | | Rₚ-RVX | | fast^{a} | | slow^{a} | GA | |
| 0 | PTE S5^{b} | 6.8 × 10⁻³ ± | 2 × 10⁻⁴ | 0.02 ± | 1 × 10⁻³ | N.D^{c} | | N.D^{c} | 690 ± | 140 |
| 5 | C23^{b} | 0.70 ± | 0.1 | 0.30 ± | 0.03 | 0.20 ± | 0.02 | N.D^{c} | 158 ± | 18 |

| 10 | C23- | 0.80 ± | 0.2 | 5.90 ± | 0.3 | N.D^{c} | | N.D^{c} | | N.D^{c} |
|---|---|---|---|---|---|---|---|---|---|---|
| | Y309W | | | | | | | | | |
| 11 | C23- | 0.8 ± | 0.1 | 4.5 ± | 0.8 | 7.5 ± | 1.4 | 1.9 ± | 0.8 | N.D^{c} |
| | Y309W- | | | | | | | | | |
| | m2p0 | | | | | | | | | |
| 11 | 4E11 | 0.70 ± | 0.1 | 0.50 ± | 0.1 | 1.10 ± | 0.1 | 0.4 ± | 0.03 | N.D^{c} |
| 12 | IVA1- | 0.5 ± | 0.1 | 4.6 ± | 0.6 | 7.5 ± | 1.4 | 1.9 ± | 0.8 | N.D^{c} |
| | m2p0 | | | | | | | | | |
| 13 | 1-3-D5 | N.D^{c} | | N.D^{c} | | N.D^{c} | | N.D^{c} | | N.D^{c} |
| 13 | 10-2-C3 | 2.5 ± | 0.1 | 0.9 ± | 0.1 | 3.7 ± | 0.2 | 1.8 ± | 0.6 | N.D^{c} |
| 13 | 10-1-D11 | 2.0 ± | 0.2 | 0.7 ± | 0.01 | 3.4 ± | 0.01 | 1.1 ± | 0.1 | N.D^{c} |

| Round | Variant | | | GD- | | GD- | | | |
|---|---|---|---|---|---|---|---|---|---|
| # | name | GB | | fast | | slow | | GF | |
| 0 | PTE S5^{b} | 8.2 ± | 0.7 | 1 ± | 0.3 | 0.1 ± | 0.03 | 0.05 ± | 8 × 10⁻³ |
| 5 | C23^{b} | 148 ± | 24 | 1 ± | 31 | 0.10 ± | 0.03 | 0.05 ± | 0.01 |
| 10 | C23- | N.D^{c} | | N.D^{c} | | N.D^{c} | | N.D^{c} | |
| | Y309W | | | | | | | | |
| 11 | C23- | N.D^{c} | | N.D^{c} | | N.D^{c} | | N.D^{c} | |
| | Y309W- | | | | | | | | |
| | m2p0 | | | | | | | | |
| 11 | 4E11 | N.D^{c} | | 5.1 ± | 1.7 | 0.75 ± | 0.01 | 3.5 ± | 0.1 |
| 12 | IVA1- | N.D^{c} | | 3.8 ± | 0.04 | 1.1 ± | 0.1 | 1.5 ± | 0.1 |
| | m2p0 | | | | | | | | |
| 13 | 1-3-D5 | N.D^{c} | | N.D^{c} | | N.D^{c} | | 1 ± | 0.2 |
| 13 | 10-2-C3 | 83 ± | 22 | 1.4 ± | 0.1 | 0.2 ± | 0.02 | 3 ± | 0.6 |
| 13 | 10-1-D11 | 55 ± | 7.5 | 0.7 ± | 0.1 | 0.7 ± | 0.1 | 3.2 ± | 1.1 |

^{a}The CVX isomers were not separated for individual analysis as Rp and Sp, and are only identified by their rate of hydrolysis.
^{b}Taken from (Cherny, et al., 2013)
^{c}N.D—not determined

The second-order rate constant for inhibition of hAChE by the Sₚ isomer of VX (kᵢ=1.4×10⁸ M⁻¹ min⁻¹) is 116 fold greater than that of its Rₚ isomer (Ordentlich, et al., 2004). Thus, it is sufficient for a catalytic bioscavenger to have an efficiency (k_{cat}K_{M}) that is ≥5×10⁵ M⁻¹ min⁻¹ with the Rₚ isomer of VX, in order to detoxify this isomer in the circulation before it inhibits blood cholinesterase activity. It appears that the present most active, evolved variants amply meet this threshold criterion (Table 4).

CVX is similar in structure to RVX except for its O-alkyl group. A previous analysis of selected variants from rounds 1-7 indicated that the toxic Sₚ isomers of CVX and RVX were hydrolyzed at very similar rates (Goldsmith, et al., 2015). Indeed, evolved variants from rounds 11-13, exhibited similar catalytic efficiencies with Sₚ-RVX and the rapidly hydrolyzed isomer of CVX (Tables 2, 4). Thus, the present most improved RVX hydrolyzing variant, IVA1-m2pO, may also provide effective protection from CVX.

G-type nerve agents are considered very toxic OPs and some of them are refractory to standard oxime therapy. Previously, it was found that some G-agents such as GA and GB were efficiently hydrolyzed by PTE variants that were evolved towards VX (Cherny, et al., 2013). Here, the present inventors tested for the ability of our evolved variants to hydrolyze all G-type nerve agents and found that they hydrolyze the toxic isomers of GF and GD with ˜10-200-fold lower rates than V-agents (Table 4). GA and GB, on the other hand, were hydrolyzed at rates that far exceed the catalytic efficiency goal (5.5-28×10⁷ M⁻¹ min⁻¹, Table 4). Overall, as discussed below, it appears that a combination of two PTE variants could cover the entire spectrum of V-agents, GA and GB with k_{cat}/K_{M} values in the range of 1-5×10⁷ M⁻¹ min⁻¹.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

Althoff E. A., Wang L., Jiang L., Giger L., Lassila J. K., Wang Z., Smith M., Hari S., Kast P., Herschlag D. et al. (2012) Robust design and optimization of retroaldol enzymes. Protein Sci, 21, 717-726. First published on, doi: 10.1002/pro.2059.
Aricescu A. R., Lu W. and Jones E. Y. (2006) A time- and cost-efficient system for high-level protein production in mammalian cells. Acta Crystallogr D Biol Crystallogr, 62, 1243-1250. First published on, doi: 10.1107/S0907444906029799.
Ashani Y., Goldsmith M., Leader H., Wieczorek G., Silman I., Sussman J. L. and Tawfik D. S. (2013) The ‘thio effect’—a kinetic barrier in OPH-catalyzed hydrolysis of P—S—R containing OPs. Febs J, 280, 167-167. First published on.
Ashani Y., Leader H., Aggarwal N., Silman I., Worek F., Sussman J. L. and Goldsmith M. (2016) In vitro evaluation of the catalytic activity of paraoxonases and phosphotriesterases predicts the enzyme circulatory levels required for in vivo protection against organophosphate intoxications. Chem Biol Interact. First published on, doi: 10.1016/j.cbi.2016.04.039.
Ashani Y. and Pistinner S. (2004) Estimation of the upper limit of human butyrylcholinesterase dose required for protection against organophosphates toxicity: a mathematically based toxicokinetic model. Toxicol Sci, 77, 358-367. First published on.
Aubert S. D., Li Y. and Raushel F. M. (2004) Mechanism for the hydrolysis of organophosphates by the bacterial phosphotriesterase. Biochemistry, 43, 5707-5715.
First published on, doi: 10.1021/bi0497805.
Bar-Even A., Noor E., Savir Y., Liebermeister W., Davidi D., Tawfik D. S. and Milo R. (2011) The moderately efficient enzyme: evolutionary and physicochemical trends shaping enzyme parameters. Biochemistry, 50, 4402-4410. First published on, doi: 10.1021/bi2002289.
Bigley A. N., Xu C. F., Henderson T. J., Harvey S. P. and Raushel F. M. (2013) Enzymatic Neutralization of the Chemical Warfare Agent VX: Evolution of Phosphotriesterase for Phosphorothiolate Hydrolysis. J Am Chem Soc, 135, 10426-10432. First published on, doi: DOI 10.1021/ja402832z.
Bird S. B., Dawson A. and Ollis D. (2010) Enzymes and bioscavengers for prophylaxis and treatment of organophosphate poisoning. Front Biosci (Schol Ed), 2, 209-220. First published on.
Bloom J. D., Labthavikul S. T., Otey C. R. and Arnold F. H. (2006) Protein stability promotes evolvability. Proc Natl Acad Sci USA, 103, 5869-5874. First published on, doi: 10.1073/pnas.0510098103.
Cherny I., Greisen P., Jr., Ashani Y., Khare S. D., Oberdorfer G., Leader H., Baker D. and Tawfik D. S. (2013) Engineering V-type nerve agents detoxifying enzymes using computationally focused libraries. ACS chemical biology, 8, 2394-2403. First published on, doi: 10.1021/cb4004892.
Chou H. H., Chiu H. C., Delaney N. F., Segre D. and Marx C. J. (2011) Diminishing returns epistasis among beneficial mutations decelerates adaptation. Science, 332, 1190-1192. First published on, doi: 10.1126/science. 1203799.
Currin A., Swainston N., Day P. J. and Kell D. B. (2015) Synthetic biology for the directed evolution of protein biocatalysts: navigating sequence space intelligently. Chemical Society Reviews, 44, 1172-1239. First published on, doi: 10.1039/C4CS00351A.
Davis I. W. and Baker D. (2009) RosettaLigand docking with full ligand and receptor flexibility. J Mol Biol, 385, 381-392. First published on, doi: 10.1016/j.jmb.2008.11.010.
Dellus-Gur E., Elias M., Caselli E., Prati F., Salverda M. L., de Visser J. A., Fraser J. S. and Tawfik D. S. (2015) Negative Epistasis and Evolvability in TEM-1 beta-Lactamase—The Thin Line between an Enzyme's Conformational Freedom and Disorder. J Mol Biol, 427, 2396-2409. First published on, doi: 10.1016/j.jmb.2015.05.011.
Ellman G. L., Courtney K. D., Andres V., Jr. and Feather-Stone R. M. (1961) A new and rapid colorimetric determination of acetylcholinesterase activity. Biochem Pharmacol, 7, 88-95. First published on.
Fasan R., Chen M. M., Crook N. C. and Arnold F. H. (2007) Engineered alkane-hydroxylating cytochrome P450(BM3) exhibiting nativelike catalytic properties. Angew Chem Int Ed Engl, 46, 8414-8418. First published on, doi: 10. 1002/anie.200702616.
Fleishman S. J., Leaver-Fay A., Corn J. E., Strauch E. M., Khare S. D., Koga N., Ashworth J., Murphy P., Richter F., Lemmon G. et al. (2011) RosettaScripts: a scripting language interface to the Rosetta macromolecular modeling suite. PLoS One, 6, e20161. First published on, doi: 10.1371/journal.pone.0020161.
Frommer W. B., Davidson M. W. and Campbell R. E. (2009) Genetically encoded biosensors based on engineered fluorescent proteins. Chem Soc Rev, 38, 2833-2841. First published on, doi: 10.1039/b907749a.
Fuhrmann G. and Leroux J.-C. (2014) Improving the Stability and Activity of Oral Therapeutic Enzymes—Recent Advances and Perspectives. Pharmaceutical Research, 31, 1099-1105. First published on, doi: 10.1007/s11095-013-1233-y.
Girvan H. M. and Munro A. W. (2016) Applications of microbial cytochrome P450 enzymes in biotechnology and synthetic biology. Curr Opin Chem Biol, 31, 136-145.
First published on, doi: 10.1016/j.cbpa.2016.02.018.
Goldenzweig A., Goldsmith M., Hill S. E., Gertman O., Laurino P., Ashani Y., Dym O., Unger T., Albeck S., Prilusky J. et al. (2016) Automated Structure- and Sequence-Based Design of Proteins for High Bacterial Expression and Stability. Mol Cell, 63, 337-346. First published on, doi: 10.1016/j.molcel.2016.06.012.
Goldsmith M., Ashani Y., Simo Y., Ben-David M., Leader H., Silman I., Sussman J. L. and Tawfik D. S. (2012) Evolved stereoselective hydrolases for broad-spectrum g-type nerve agent detoxification. Chem Biol, 19, 456-466. First published on 2012 Apr. 24, doi: 10.1016/j.chembiol.2012.01.017.
Goldsmith M., Eckstein S., Ashani Y., Greisen P., Jr., Leader H., Sussman J. L., Aggarwal N., Ovchinnikov S., Tawfik D. S., Baker D. et al. (2015) Catalytic efficiencies of directly evolved phosphotriesterase variants with structurally different organophosphorus compounds in vitro. Archives of toxicology. First published on, doi: 10.1007/s00204-015-1626-2.
Grass G., Fan B., Rosen B. P., Franke S., Nies D. H. and Rensing C. (2001) ZitB (YbgR), a member of the cation diffusion facilitator family, is an additional zinc transporter in Escherichia coli. J Bacteriol, 183, 4664-4667. First published on, doi: 10.1128/JB.183.15.4664-4667.2001.
Gupta R. D., Goldsmith M., Ashani Y., Simo Y., Mullokandov G., Bar H., Ben-David M., Leader H., Margalit R., Silman I. et al. (2011) Directed evolution of hydrolases for prevention of G-type nerve agent intoxication. Nat Chem Biol, 7, 120-125. First published on 2011 Jan. 11, doi: 10. 1038/nchembio.510.
Jacquet P., Daude D., Bzdrenga J., Masson P., Elias M. and Chabriere E. (2016) Current and emerging strategies for organophosphate decontamination: special focus on hyperstable enzymes. Environ Sci Pollut Res Int, 23, 8200-8218. First published on, doi: 10.1007/s11356-016-6143-1.
Jemli S., Ayadi-Zouari D., Hlima H. B. and Bejar S. (2016) Biocatalysts: application and engineering for industrial purposes. Crit Rev Biotechnol, 36, 246-258. First published on, doi: 10.3109/07388551.2014.950550.
Kapust R. B. and Waugh D. S. (1999) Escherichia coli maltose-binding protein is uncommonly effective at promoting the solubility of polypeptides to which it is fused. Protein Science: A Publication of the Protein Society, 8, 1668-1674. First published on.
Karigar C. S. and Rao S. S. (2011) Role of microbial enzymes in the bioremediation of pollutants: a review. Enzyme Res, 2011, 805187. First published on, doi: 10.4061/2011/805187.
Kaur J. and Sharma R. (2006) Directed evolution: an approach to engineer enzymes. Crit Rev Biotechnol, 26, 165-199. First published on, doi: 10.1080/07388550600851423.
Kellogg E. H., Leaver-Fay A. and Baker D. (2011) Role of conformational sampling in computing mutation-induced changes in protein structure and stability. Proteins, 79, 830-838. First published on, doi: 10.1002/prot.22921.
Khare S. D., Kipnis Y., Greisen P., Jr., Takeuchi R., Ashani Y., Goldsmith M., Song Y., Gallaher J. L., Silman I., Leader H. et al. (2012) Computational redesign of a mononuclear zinc metalloenzyme for organophosphate hydrolysis. Nat Chem Biol, 8, 294-300. First published on, doi: 10.1038/nchembio.777.
Kotzia G. A. and Labrou N. E. (2009) Engineering thermal stability of L-asparaginase by in vitro directed evolution. FEBS J, 276, 1750-1761. First published on, doi: 10.1111/j.1742-4658.2009.06910.x.
Kuhlman B., Dantas G., Ireton G. C., Varani G., Stoddard B. L. and Baker D. (2003) Design of a novel globular protein fold with atomic-level accuracy. Science, 302, 1364-1368. First published on, doi: 10.1126/science. 1089427.
Leaver-Fay A., O'Meara M. J., Tyka M., Jacak R., Song Y., Kellogg E. H., Thompson J., Davis I. W., Pache R. A., Lyskov S. et al. (2013) Scientific benchmarks for guiding macromolecular energy function improvement. Methods Enzymol, 523, 109-143. First published on, doi: 10.1016/B978-0-12-394292-0.00006-0.
Lenz D. E., Yeung D., Smith J. R., Sweeney R. E., Lumley L. A. and Cerasoli D. M. (2007) Stoichiometric and catalytic scavengers as protection against nerve agent toxicity: a mini review. Toxicology, 233, 31-39. First published on.
Liu Y., Matharu Z., Howland M. C., Revzin A. and Simonian A. L. (2012) Affinity and enzyme-based biosensors: recent advances and emerging applications in cell analysis and point-of-care testing. Anal Bioanal Chem, 404, 1181-1196. First published on, doi: 10.1007/s00216-012-6149-6.
MacLean R. C., Perron G. G. and Gardner A. (2010) Diminishing returns from beneficial mutations and pervasive epistasis shape the fitness landscape for rifampicin resistance in Pseudomonas aeruginosa. Genetics, 186, 1345-1354. First published on, doi: 10.1534/genetics. 110.123083.
McGuffin L. J., Bryson K. and Jones D. T. (2000) The PSIPRED protein structure prediction server. Bioinformatics, 16, 404-405. First published on.
Meiler J. and Baker D. (2006) ROSETTALIGAND: protein-small molecule docking with full side-chain flexibility. Proteins, 65, 538-548. First published on, doi: 10. 1002/prot.21086.
Munro N. (1994) Toxicity of the organophosphate chemical warfare agents GA, GB, and VX: implications for public protection. Environ Health Perspect, 102, 18-38. First published on.
Obexer R., Pott M., Zeymer C., Griffiths A. D. and Hilvert D. (2016) Efficient laboratory evolution of computationally designed enzymes with low starting activities using fluorescence-activated droplet sorting. Protein Eng Des Sel, 29, 355-366. First published on, doi: 10.1093/protein/gzw032.
Ordentlich A., Barak D., Sod-Moriah G., Kaplan D., Mizrahi D., Segall Y., Kronman C., Karton Y., Lazar A., Marcus D. et al. (2004) Stereoselectivity toward VX is determined by interactions with residues of the acyl pocket as well as of the peripheral anionic site of AChE. Biochemistry, 43, 11255-11265. First published on, doi: 10.1021/bi0490946.
Porter J. L., Rusli R. A. and Ollis D. L. (2016) Directed Evolution of Enzymes for Industrial Biocatalysis. Chembiochem, 17, 197-203. First published on, doi: 10.1002/cbic.201500280.
Rockah-Shmuel L. and Tawfik D. S. (2012) Evolutionary transitions to new DNA methyltransferases through target site expansion and shrinkage. Nucleic Acids Res, 40, 11627-11637. First published on, doi: 10.1093/nar/gks944.
Rockah-Shmuel L., Tawfik D. S. and Goldsmith M. (2014) Generating targeted libraries by the combinatorial incorporation of synthetic oligonucleotides during gene shuffling (ISOR). Methods Mol Biol, 1179, 129-137. First published on, doi: 10.1007/978-1-4939-1053-3_8.
Rokyta D. R., Joyce P., Caudle S. B., Miller C., Beisel C. J. and Wichman H. A. (2011) Epistasis between beneficial mutations and the phenotype-to-fitness Map for a ssDNA virus. PLoS Genet, 7, e1002075. First published on, doi: 10.1371/journal.pgen. 1002075.
Roodveldt C. and Tawfik D. S. (2005) Directed evolution of phosphotriesterase from Pseudomonas diminuta for heterologous expression in Escherichia coli results in stabilization of the metal-free state. Protein engineering, design & selection: PEDS, 18, 51-58. First published on 2005 Mar. 26, doi: 10.1093/protein/gzi005.
Salverda M. L., Dellus E., Gorter F. A., Debets A. J., van der Oost J., Hoekstra R. F., Tawfik D. S. and de Visser J. A. (2011) Initial mutations direct alternative pathways of protein evolution. PLoS Genet, 7, e1001321. First published on, doi: 10.1371/journal.pgen. 1001321.
Schellekens H. (2002) Immunogenicity of therapeutic proteins: clinical implications and future prospects. Clin Ther, 24, 1720-1740; discussion 1719. First published on.
Schenk M. F., Szendro I. G., Salverda M. L., Krug J. and de Visser J. A. (2013) Patterns of Epistasis between beneficial mutations in an antibiotic resistance gene. Mol Biol Evol, 30, 1779-1787. First published on, doi: 10.1093/molbev/mst096.
Schoustra S., Hwang S., Krug J. and de Visser J. A. (2016) Diminishing-returns epistasis among random beneficial mutations in a multicellular fungus. Proc Biol Sci, 283. First published on, doi: 10.1098/rspb.2016.1376.
Sikosek T. and Chan H. S. (2014) Biophysics of protein evolution and evolutionary protein biophysics. J R Soc Interface, 11, 20140419. First published on, doi: 10. 1098/rsif.2014.0419.
Songa E. A. and Okonkwo J. O. (2016) Recent approaches to improving selectivity and sensitivity of enzyme-based biosensors for organophosphorus pesticides: A review. Talanta, 155, 289-304. First published on, doi: 10.1016/j.talanta.2016.04.046.
Sussman J. L., Harel M., Frolow F., Varon L., Toker L., Futerman A. H. and Silman I. (1988) Purification and crystallization of a dimeric form of acetylcholinesterase from Torpedo californica subsequent to solubilization with phosphatidylinositol-specific phospholipase C. J Mol Biol, 203, 821-823. First published on.
Taheri M. N., Behzad-Behbahani A., Rafiei Dehbidi G., Salehi S. and Sharifzadeh S. (2016) Engineering, expression and purification of a chimeric fibrin-specific streptokinase. Protein Expr Purif 128, 14-21. First published on, doi: 10.1016/j.pep.2016.08.003.
Tokuriki N., Jackson C. J., Afriat-Jurnou L., Wyganowski K. T., Tang R. and Tawfik D. S. (2012) Diminishing returns and tradeoffs constrain the laboratory optimization of an enzyme. Nat Commun, 3, 1257. First published on, doi: 10. 1038/ncomms2246.
Tokuriki N. and Tawfik D. S. (2009) Stability effects of mutations and protein evolvability. Curr Opin Struct Biol, 19, 596-604. First published on, doi: 10.1016/j.sbi.2009.08.003.
Whitehead T. A., Chevalier A., Song Y., Dreyfus C., Fleishman S. J., De Mattos C., Myers C. A., Kamisetty H., Blair P., Wilson I. A. et al. (2012) Optimization of affinity, specificity and function of designed influenza inhibitors using deep sequencing. Nat Biotechnol, 30, 543-548. First published on, doi: 10.1038/nbt.2214.
Wijma H. J. and Janssen D. B. (2013) Computational design gains momentum in enzyme catalysis engineering. FEBS J, 280, 2948-2960. First published on, doi: 10.1111/febs.12324.
Wille T., Neumaier K., Koller M., Ehinger C., Aggarwal N., Ashani Y., Goldsmith M., Sussman J. L., Tawfik D. S., Thiermann H. et al. (2016) Single treatment of VX poisoned guinea pigs with the phosphotriesterase mutant C23AL: Intraosseous versus intravenous injection. Toxicol Lett, 258, 198-206. First published on, doi: 10. 1016/j.toxlet.2016.07.004.
Worek F., Seeger T., Goldsmith M., Ashani Y., Leader H., Sussman J. S., Tawfik D., Thiermann H. and Wille T. (2014) Efficacy of the rePON1 mutant IIG1 to prevent cyclosarin toxicity in vivo and to detoxify structurally different nerve agents in vitro. Archives of toxicology, 88, 1257-1266. First published on, doi: 10.1007/s00204-014-1204-z.
Worek F., Seeger T., Reiter G., Goldsmith M., Ashani Y., Leader H., Sussman J. L., Aggarwal N., Thiermann H. and Tawfik D. S. (2014) Post-exposure treatment of VX poisoned guinea pigs with the engineered phosphotriesterase mutant C23: A proof-of-concept study. Toxicology Letters, 231, 45-54.
Worek F., Thiermann H. and Wille T. (2016) Catalytic bioscavengers in nerve agent poisoning: A promising approach? Toxicol Lett, 244, 143-148. First published on, doi: 10. 1016/j.toxlet.2015.07.012.
Yang G. and Withers S. G. (2009) Ultrahigh-throughput FACS-based screening for directed enzyme evolution. Chembiochem, 10, 2704-2715. First published on, doi: 10.1002/cbic.200900384.
Zheng F., Xue L., Hou S., Liu J., Zhan M., Yang W. and Zhan C. G. (2014) A highly efficient cocaine-detoxifying enzyme obtained by computational design. Nat Commun, 5, 3457. First published on, doi: 10.1038/ncomms4457.

1. A polypeptide comprising an amino acid sequence of phosphotriesterase (PTE), wherein said amino acid sequence comprises the mutations K77A, A80V/M, F132E, T173N, G208D, D233G/N/M/L/R/V/A/S/T, H254G, I274N and Y309W, wherein the numbering of the mutations is according to PDB 1HZY crystal structure numbering, wherein said polypeptide has at least 2500 fold the catalytic efficiency for a VX-type nerve agent as a polypeptide which consists of the sequence as set forth in SEQ ID NO: 1, when assayed at 25° C. under identical conditions and at least 3000 fold the catalytic efficiency for a RVX-type nerve agent as a polypeptide which consists of the sequence as set forth in SEQ ID NO: 1, when assayed at 25° C. under identical conditions.

2. The polypeptide of claim 1, wherein said amino acid sequence comprises the mutation D233G.

3. The polypeptide of claim 1, wherein the amino acid at position 106 is isoleucine.

4. The polypeptide of claim 1, having at least 10 fold the catalytic efficiency for a VX-type nerve agent as a polypeptide which consists of the sequence as set forth in SEQ ID NO: 31, when assayed at 25° C. under identical conditions.

5. The polypeptide of claim 4, comprising the mutations A80M, A270S, L271W and D233G.

6. The polypeptide of claim 5, comprising a PTE amino acid sequence at least 99% homologous to the sequence as set forth in SEQ ID NO: 9, 19 or 40.

7. The polypeptide of claim 4, comprising the mutations A80M, S267M, A270S, D233G and L271W.

8. The polypeptide of claim 7, comprising a PTE amino acid sequence at least 99% homologous to the sequence as set forth in SEQ ID NOs: 11, 21 or 41.

9. The polypeptide of claim 1, comprising the mutations K77A, A80V, F132E, T173N, G208D, D233G, H254G, I274N and Y309W.

10. The polypeptide of claim 9, comprising a PTE amino acid sequence at least 99% homologous to the sequence as set forth in SEQ ID NOs: 7, 17 or 39.

11. A polypeptide comprising an amino acid sequence of phosphotriesterase (PTE) having at least 8,000 fold the catalytic efficiency for a RVX-type nerve agent as a polypeptide which consists of the sequence as set forth in SEQ ID NO: 1, when assayed at 25° C. under identical conditions, wherein said amino acid sequence comprises the mutations K77A, A80V/M, I106A, F132E, T173N, G208D, H254G, I274N, Y309W and D233G/N/M/L/R/V/A/S/T, wherein the numbering of the mutations is according to PDB 1HZY crystal structure numbering.

12. The polypeptide of claim 11, further comprising the mutations A80V, C59M/V/F and an A266 deletion.

13. The polypeptide of claim 12, further comprising the mutations A80V, C59M and an A266 deletion.

14. (canceled)

15. The polypeptide of claim 11, further comprising the mutations A80M, S267M, A270S and L271W.

16. (canceled)

17. The polypeptide of claim 1, further comprising at least one of the stabilizing mutations selected from the group consisting of R118E, A203D, S222D, S238D, M293V/A, G348T and T352E/D.

18. The polypeptide of claim 1, further comprising each of the stabilizing mutations R118E, A203D, S222D, S238D, M293V, G348T and T352E/D.

19. (canceled)

20. The polypeptide of claim 1, comprising an amino acid sequence at least 99% homologous to the sequence as set forth in SEQ ID NO: 7, 17, 9, 19, 11, 21, 39, 40 or 41.

21. The polypeptide of claim 11, comprising an amino acid sequence at least 99% homologous to the sequence as set forth in SEQ ID NO: 5, 15, 13, 23, 25, 26, 27, 28, 38, 42, 43 or 44.

22-27. (canceled)

28. A method of treating an organophosphate exposure associated damage in a subject, comprising administering to the subject a therapeutically effective amount of the polypeptide of claim 1.

29. An article of manufacture for treating or preventing organophosphate exposure associated damage, the article of manufacture comprising the polypeptide of claim 1 immobilized on to a solid support.

30-33. (canceled)
